(19) United States
(12) Patent Application Publication
(10) Pub. No.: US 2006/0147906 A1
(43) Pub. Date: Jul. 6, 2006

(54) Anti-hpv-16 e7 antibodies and their use

(75) Inventors: Werner Paul Zwerschke, Innsbruck (AT); Pidder Jansen-Duerr, Innsbruck (AT); Marc Fiedler, Innsbruck (AT); Johann Peter Viertler, Mieders (AT); Andreas Widschwendter, Jenbach (AT); Elisabeth Mueller-Holzner, Gnadenwald (AT); Andreas Laich, Innsbruck (AT); Barhard Fitzky, Arams (AT)

Correspondence Address: COOLEY GODWARD LLP;ATTN: PATENT GROUP, 11951 FREEDOM DRIVE, SUITE 1600, ONE FREEDOM SQUARE- RESTON TOWN CENTER, RESTON, VA, 20190-5656, US

(73) Assignee: Amynon Biotech GmbH, Innsbruck (AT)

(21) Appl. No.: 10/508,766

(22) PCT Filed: Mar. 21, 2003
(86) PCT No.: PCT/EP03/02990

§ 371 Date: Sep. 28, 2005

(30) Foreign Application Priority Data
Mar. 22, 2002 (EP) 02 00 6490.3
Sep. 27, 2002 (EP) 02 02 1687.5
Feb. 21, 2003 (EP) 03 00 3957.2

(51) Int. Cl. C12Q 1/70; C07K 16/08

(52) U.S. Cl. 435/5; 530/388.3

(57) ABSTRACT

The present invention relates to an anti-HPV-16 E7 antibody obtainable by (a) eliciting an in vivo humoral response against highly purified HPV-16 E7 protein or a fragment thereof in a non-human vertebrate; and (b) affinity-purifying antibodies as obtained in the eliciting-step (a). Furthermore, the invention provides for the use of the anti-HPV-1 E7 antibody/antibodies for the preparation of a diagnostic composition of the (immuno-)histological detection of expressed HPV-16 E7 in a biological sample. Additionally, the invention relates to specific diagnostic compositions as well as to methods producing the same. The invention also provides for kits comprising (an) anti-HPV-16 E7 antibody(ies) of the invention or a diagnostic composition and discloses in vitro methods for the detection of a sexually transmittable disease or cancer, in particular cervical cancer, breast cancer, prostate cancer, anogenital cancer/neoplasia, penile cancer or head and neck cancer by using the described antibodies.

The present invention relates to an anti-HPV-16 E7 antibody obtainable by (a) eliciting an in vivo humoral response against highly purified HPV-16 E7 protein or a fragment thereof in a non-human vertebrate; and (b) affinity-purifying antibodies as obtained in the eliciting-step (a). Furthermore, the invention provides for the use of the anti-HPV-16 E7 antibody/antibodies for the preparation of a diagnostic composition for the (immuno-) histological detection of expressed HPV-16 E7 in a biological sample. Additionally, the invention relates to diagnostic compositions as well as to methods for producing the same. The invention also provides for kits comprising (an) anti-HPV-16 E7 antibody(ies) of the invention or a diagnostic composition of the invention and discloses in vitro methods for the detection of a sexually transmittable disease or cancer, in particular cervical cancer, breast cancer, prostate cancer, head and neck cancer or anogenital cancer by using the described antibodies.

Despite an intensive screening program, cervical cancer is one of the most predominant neoplastic diseases in women with a world-wide, incidence second only to breast cancer (Walboomers, 1999). A major etiological factor in the genesis of cervical carcinoma is the infection by human papillomaviruses (HPVs), which are small DNA viruses that infect epithelial cells of either the skin or mucosa. Of the almost 100 different types of HPV that have been characterised to date, approximately two dozen specifically infect genital and oral mucosa (reviewed in zur Hausen, 2000). On the basis of epidemiological and biochemical data, HPVs are subdivided into two groups. Genital HPVs of the high-risk group (most commonly HPV-16, 18) cause cervical cancer and other anogenital cancers while papillomaviruses of the low-risk group (most frequently HPV-6, 11) cause benign genital warts (for review, see Howley, 1996). PCR based studies have shown that more than 99% of invasive cervical cancers world-wide contain high risk HPVs (Walboomers, 1999); however, malignant progression occurs only in a small subset of infected patients and is typically slow (reviewed in Alexander and Phelps, 2000). Most cervical dysplasia represent squamous cell carcinoma and the main diagnostic tool to detect cervical dysplastic cells is still based on cytological screening using Pap-smear analysis introduced in 1943 by Papanicolaou (1942) (for review, see Koss, 1989; Meijer and Walboomers, 2000). Although Pap-smear analysis has proven highly effective it is difficult to standardise, which is reflected by a high false negative error rate of approximately 30% (Walboomers, 1995; Clavel, 1999; Renshaw et al., 2001). Despite the introduction of mass screening programs, the best of which have dropped the mortality rates by 70%, incidence of cervical cancer in the United States has been increasing by about 3% a year since 1986 in spite of an intensification of the rate of screening (Larsen, 1994).

Since it is medical general knowledge that cervical cancer arises as consequence of persistent high-risk papillomavirus infections (reviewed in zur Hausen, 2000), the problem could be addressed by the introduction of HPV tests into screening programmes for better identification of patients at risk. At present, for clinical applications, PCR and the hybrid capture analysis, both are DNA-detecting methods, are only useful to a limited extend (reviewed in Milde-Langosch, 2000). Milde-Langosch furthermore teaches that, besides the molecular biological methods, some antibodies against early HPV-proteins are sold (for example by Santa Cruz Biotechnology or Dianova) but these antibodies are, in comparison to these molecular biological techniques even less sensitive in (immuno)-histochemical analysis.

The major disadvantage of the above discussed molecular biology methods systems is, that they do only allow to detect viral infection, however, about 5-30% of the normal female population harbours these viruses and only very few of these develop clinically relevant lesions (von Knebel Doeberitz, 2001). In accordance with this consideration, a high rate of transient and asymptomatic HPV infections was found especially among young woman (Schiffman and Brinton, 1995). Given the low incidence of cervical cancer, it may not be useful to apply HPV detection for cervical cancer screening in this age group. Moreover, the PCR based screening systems, although highly sensitive, are not widely applied for the reason that HPV DNA will be detected in a wide range of normal cytological smears resulting in a high rate of false positive amplification (reviewed in Trofatter, 1997).

It is well established that the expression of E6 and E7, in epithelial stem cells of the mucosa, is required to initiate and maintain cervical carcinogenesis (for recent review, see Mantovani and Banks, 2001; Münger, 2001). Thus, a promising way to improve the screening programs could be to measure the expression of the E6 and E7 oncoprotein which initiate in a long term process neoplastic transformation in few of the HPV harbouring cells. Since these viral proteins are not expressed in normal cervical squamous epithelia, screening for high risk E7 over-expressing cells allows to specifically identify dysplastic lesions. Moreover, progression of pre-neoplastic lesions to invasive cervical cancers is often associated with a continuous enhanced expression of the E6 and E7 oncoprotein (Schwarz, 1985; Francis, 2000). Similar to these considerations, Klaes (2001) monitored the overexpression of the cyclin-dependent kinase inhibitor p16 (INK4a), a gene which is upregulated in response to E7, as a marker for dysplastic and neoplastic epithelial cells of the cervix uteri. However, p16 (INK4A) is only one of several genes which are upregulated in response to E7 (for review, see McMurray, 2001) and upregulation of p16 (INK4A) expression is not necessary for E7 induced malignant transformation (Giarre, 2001). Furthermore, in view of its central role as tumorsuppressor and cell cycle inhibitory protein p16 (INK4A) is upregulated by several other, growth suppressing, stimuli, thus p16 (INK4a) is for example well known as target of senescence-inducing pathways (for review, see Bringold and Serrano, 2000). Consequently upregulation of p16 (INK4A) might not necessarily reflect the activity of the E7 oncoprotein.

Furthermore, some studies have speculated on the prevalence of anogenital types of human papillomavirus in prostate cancer and benign prostate hypertrophy. Interestingly, the prevalence of an HPV, in particular HPV-16 infection in prostate carcinogenesis is highly disputed. Cuzick (1995, Cancer Sarr. 23, 91-95) review earlier reports on this issue and stress that it is unlikely that common anogenital papillomaviruses have an important role in prostate carcinogenesis. Several studies have linked the presence of HPV16 DNA to a risk for developing prostate cancer (e.g. Moyret-Lalle, 1997, Int. J. Cancer 64, 125-129; Jerris, 1997 Urology 50, 150-156 or Suzuki, 1996, Prostate 28, 318-324), yet, these studies did not provide for a conclusive relationship between prostate cancerogenesis and HPV-16 activities and/or the expression of HPV16 proteins. Wideroff (1996, Prostate 28, 117-123) even teaches that HPV infection is not a significant risk factor for prostate cancer and Anderson (1997, J. Med. Virol. 52, 8-13) confirms the teaching that HPV16 and closely related types are unlikely initiations of prostate cancer. Similarly Noda (1998, Urol. Res. 26, 165-169) suggests that HPV is not a causal factor for prostatic cancer or benign prostatic hyperplasia, and Stickler (1998, Cancer 82, 1118-1125 and 1998, Eur. J. Canc. Prev. 7, 305-313) comes to the conclusion that HPV is not associated with prostate carcinomas. Even if Serth (1998) analysed by single-tube quantitative, competitive PCR samples from prostate cancers and indicates that in accordance with this DNA-detection method, HPV16 might contribute to the development of a subset of prostate cancers (Serth (1999), Canc. Res. 59, 823-825), another study of the same year (Saad (1999), Can. J. Urol. 6, 834-838) could not detect HPV DNA in fresh tissue from patients undergoing radical prostatectomy for prostate cancer. Accordingly, the role of HPVs, in particular HPV16, in prostate cancer remains controversial and elusive.

Several sets of monoclonal antibodies against the HPV-16 E7 oncoprotein or HPV-16 E7 derived peptides have been produced (Sato, 1989; Tindle, 1990; Selvey, 1992; Stacey, 1994; Fujikawa, 1994; Zatsepina, 1997) and commercial preparations are also available (Zymed Laboratories, San Francisco, Calif., USA; Santa Cruz Biotechnology, Santa Cruz, Calif., USA). No antibody, however, was reported as sufficient in sensitivity and specificity to recognise HPV-16 E7 neither in cytological smears nor in paraffin embedded sections from biopsies of cervical cancer patients; see Milde-Langosch, 1999. Di Lonardo (2001, Arch Viral 146, 117-125) has produced egg yolk antibodies as well as rabbit antibodies against E7 oncogenic protein of HPV16. Di Lonardo (2001), loc. cit. stresses that some commercial preparations of anti-E7 antibodies are available, but they suffer severe disadvantages and are not suitable for diagnostic purposes. Yet, the data provided by Di Lonardo (2001) are not conclusive since merely the hen antibodies were able to localize HPV-16 E7 in a cultured cell line and in a SIL (Squameous Interepithelial lesion) biopsy. However, Di Lonardo (2001) loc. cit. also teaches that the rabbit antibodies are not able to detect E7 in immunocytochemistry and stressed that the generated hen antibodies were in a number of cases unable to detect E7 protein in immunostainings of cervical lesions.

Documentation that certain high-risk types of human papillomavirus (HPV) are necessary in the etiology of cervical cancer does it make conceivable to introduce HPV based tests into screening programmes for better identification of patients at risk. Thus it is well established that the expression of the E6 and E7 genes, in epithelial stem cells of the mucosa is required to initiate and maintain cervical carcinogenesis. For this reason, a promising way to improve the screening programmes could be the measurement of the expression level of the E6 or E7 oncoprotein which initiate, in a long term process, neoplastic transformation in few of the HPV harbouring cells. These viral genes are not expressed in normal cervical squamous epithelia and the progression of pre-neoplastic lesions to invasive cervical cancers is presumably associated with a continuous enhanced expression of the E6 and E7 oncoproteins. For these reasons screening for cells overexpressing high risk E7 oncoprotein may allow to specifically grade dysplastic lesions. However, expression levels of HPV oncoproteins in cervical carcinoma are unknown so far, due to the lack of specific antisera that would recognize the viral proteins in cervical smears.

Accordingly, there is a need in the art for means and methods which provide for reproducible assays for the detection of symptomatic HPV infections for the detection of the malignant state of a cancerous cell or for the detection of a sexually transmittable disease.

Thus, the technical problem underlying the present invention is to comply with the needs described above.

In accordance, the present invention provides for an anti-HPV-16 E7 antibody obtainable by

a) eliciting an in vivo humoral response against highly purified HPV-16 E7 protein or a fragment thereof in a non-human vertebrate; and

b) affinity-purifying antibodies as obtained in the eliciting-step (a).

The term “anti-HPV-16 E7 antibody” as employed herein refers to an antibody, a plurality of antibodies and/or a serum comprising such antibodies which is/are able to specifically bind to, interact with or detect the E7 oncoprotein of HPV16 or a fragment thereof. Said term also relates to a purified serum, i.e. a purified polyclonal serum. The antibody molecule is preferably a full immunoglobulin, like an IgG, IgA, IgM, IgD, IgE, IgY (for example in yolk derived antibodies). The term “antibody” as used in this context of this invention also relates to a mixture of individual immunoglobulins. Furthermore, it is envisaged that the antibody/antibody molecule is a fragment of an antibody, like an F(ab), F(abc), Fv Fab′ or F(ab)₂. Furthermore, the term “antibody” as employed in the invention also relates to derivatives of the antibodies which display the same specificity as the described antibodies. Such derivatives may, inter alia, comprise chimeric antibodies or single-chain constructs. Yet, most preferably, said “anti-HPV-16 E7 antibody” relates to a serum, more preferably a polyclonal serum and most preferably to a purified (polyclonal) serum. The antibody/serum is obtainable, and preferably obtained, by the method described herein and illustrated in the appended examples.

The term “eliciting an in vivo humoral response in a non-human vertebrate” relates to the provocation of an immune response in a non-human vertebrate, in particular the provocation of an antibody response to HPV-16 E7 or a fragment thereof. Said antibody response comprises primary as well as secondary antibody responses to the antigenic challenge with HPV-16 E7 or a fragment thereof. The term “eliciting an in vivo humoral response”, accordingly, relates to the provocation of an immune reaction involving the production of antibodies directed towards the antigen, namely HPV-16 E7 or a fragment thereof.

The term “highly purified HPV-16 E7 protein or a fragment thereof” relates to an isolated HPV-16 E7 protein or fragment thereof, which has been purified to a purity level of at least 95%, more preferably of at least 96%, even more preferably of at least 97%, particularly preferred of at least 98% and most preferably of at least 99% purity. The purity of HPV-16 E7 protein may be confirmed by methods known in the art, preferably by densitometrical analysis as illustrated in Verdoliva (2000, J. Chormatogr. B. Biomed. Sci. Appl. 279, 233-242), Aboagye-Mathiesen (1992, Prep. Biochem. 22: 105-121) and most preferably as described in the appended examples. It is preferred that said “highly purified HPV-16 E7 protein or a fragment thereof” is purified in order to obtain the corresponding protein or fragment thereof in NMR-grade. In context of this invention, the term “highly purified HPV-16 E7 protein” relates to a purified protein E7-preparation which is at least 90%, more preferably at least 95%, more preferably at least 98% most preferably at least 99% pure. Accordingly, the highly, purified HPV-16 E7 preparation to be employed in the immunization protocols described herein comprises preferably less than 5% contaminating, unrelated proteins or protein fragments. Most preferably, said preparation comprises less than 2% contaminating, unrelated proteins or protein fragments. Purity of the highly-purified E7 preparation may be measured by methods known in the art which comprise gel stainings (in particular silver stains of SDS-PAGE followed by densitometric analysis) NMR-measurements or mass spectroscopy (MS). The purity of E7 protein or fragments thereof is in accordance with this invention, most preferably measured by analyzing samples comprising said E7 or (a) fragment(s) thereof by SDS-PAGE, followed by conventional silver staining and densitometric analysis. Corresponding protocols are detailed in the appended examples. In accordance with this invention “highly purified E7 preparations” to be employed in immunization protocols do not comprise any contaminating, unrelated proteins. According to NMR-analysis the highly-purified E7 protein (or immunogenic fragment(s) thereof) is present in a native, partially unfolded structure. Corresponding examples for such a purification is given in the appended examples. In a most preferred embodiment, the highly-purified E7-preparation is a “native, highly purifed HPV-16 E7 protein” as defined herein below. It is in particular preferred that said “native, highly purified HPV-16 E7 protein” is a full length protein, comprising preferably 98 amino acids.

The native, highly purified HPV-16 E7 protein or a fragment thereof is preferably recombinantly produced and, most preferably, said protein or fragment thereof lacks further modifications like additional tags, like His-tags or GST-tags.

In accordance with this invention, the term “native, highly purified HPV-16 E7 protein” relates to a protein which is correctly folded or relates to a stretch/fragment of said protein which is correctly folded and which is soluble, preferably highly soluble. As such, the protein is purified from E. coli under native conditions and it is not required to unfold/refold the protein by chaotropic agents, such as urea or guanidinium hydrochloride. It is in particular preferred that the native HPV-16 E7 protein comprises equivalent amounts of zinc, which is required for correct secondary structure of the E7 protein. It is of note that the term “native HPV-16 E7 protein” corresponds to the term “native, highly purified HPV-16 E7” in context of this invention and also comprises naturally occurring variants of HPV-16 E7 protein. Such variants are known in the art, as, inter alia, described by Sang Song (1997, Gynecologic Oncology 66, 275-281) or by Ku (2001), Dis. Of Colonand Rectum 44, 236-242. The person skilled in the art is easily in a position to determine the folding status of said “native HPV-16 E7 protein”, e.g. by CD analysis provided, inter alia, in the appended examples. It is envisaged, in accordance with this invention, that a native, highly purified HPV-16 E7 protein (or an immunogenic fragment thereof) is to be employed in the immunization protocols provided herein in its native, partially unfolded structure. Therefore, in purified and soluble form said E7 protein (or its immunogenic fragment) comprises, at least partially secondary structures like α-helices, β-sheets and turns and coils. In a most preferred embodiment the E7-protein to be employed in the immunization protocols provided herein comprises 7 to 8% α-helices, 45 to 47% β-sheets, 3 to 5% turns and 40 to 43% coils. The terms “β-sheet”, “α-helix”, “turn” and “coil” are very well known in the art and, inter alia, described in Brandon/Tooze (1991), “Introduction to Protein Structure”; Garland Publishing Inc., London. The HPV-16 E7 fragment to be employed in immunization protocols in accordance with this invention preferably comprise 6 to 9% α-helices, 43 to 47% β-sheets, 1 to 7% turns and/or 38 to 45% coils. In accordance with this invention it was surprisingly found that E7 protein can recombinantly be expressed and obtained in a soluble, native form as described herein. The use of highly purified recombinant E7 proteins in immunization protocols led surprisingly to high quality antibodies specific for said E7 protein. In contrast to antibodies of the prior art, the antibodies of the present invention (raised against highly purified, soluble and, preferably, native E7) are capable of specifically detecting E7 in immunobiological/immunohistochemical samples, like smears. As documented in the appended examples, prior art antibodies fail to provide for specific detection means for E7 and, accordingly, a reliable HPV diagnosis.

The term “fragment of HPV-16 E7 protein” as used herein relates to fragments of a length of at least 40, at least 50, more preferably at least 60, even more preferably at least 65 amino acid residues of the native HPV-16 E7 protein. The amino acid sequence of HPV-16 E7 and of corresponding variants is known in the art and published in Seedorf (1987, EMBO J. 6, 139-144), Sang Song (1997, loc. cit.) or Ku (2001, loc. cit.). Preferably, said fragment comprises at least the stretch of amino acids 33 to 98 of HPV-16 E7 as disclosed in Seedorf (loc. cit.). Even more preferably, however, is an E7-protein fragment that comprises at least amino acids 1 to 70 of HPV-16 E7 as disclosed in Seedorf (loc. cit.).

Preferably, the recombinantly produced HPV-16 E7 protein or its fragment is expressed in a prokaryotic host, preferably in E. coli. Yet, also other expression systems are envisaged which comprise:

Bacterial expression systems, for example, pET System, P_{L} Expression System, pCAL Vectors, pGEX Vectors, PRO Bacterial Expression System or Yeast expression systems, like pESP Vectors, pESC Vectors, Pichia Expression system, YES Vector collection, SpECTRA S. pombe Expression System, pYD1 System or Insect expression systems, like BacPAK System, Bac-to-Bac Baculovirus Expression System, Bac-N-Blue Baculovirus Expression System, DES: The Drosophila Expression System, Insectselect System or Viral expression systems, like AdEasy Adenoviral Vector System, AAV Helper-Free System ViraPort Retroviral Gene Expression System, Adeno-X Expression System, pLXSN System or Mammalian expression systems, like PMSG System, pCMV Script, pCI, Creator Gene Cloning & Expression System, Tet-On; Tet-Off Gene Expression System.

As illustrated in the appended example, preferably, said highly purified HPV-16 E7 protein or a fragment thereof is purified by a combination of ion exchange chromatography and gel filtration and said purification may further comprise, prior to ion exchange chromatography and gel filtration, a protein precipitation step.

Ion exchange chromatography is known to the artisan and ion exchange media comprise, but are not limited to Mini beads Q, Source 15 Q, Source 30 Q, Sepharose High Performance Q, Sepharose Fast Flow Q, Sepharose XL Q, Sepharose Big Beads Q, DEAE, Streamline DEAE (all from Amersham Biosciences, Vienna, Austria), DEAE-cellulose, QA-cellulose, CM-cellulose, SE-cellulose, DE-52 (Whatman, Kent, England) or Agarose based ion exchangers. Most preferably a Mono QHR 10/10 column (Amersham Biosciences, Vienna, Austria) is employed.

It is of note that also normal gravity flow or FPLC systems may be employed.

Gel filtration systems and media are also known to the skilled artisan which comprise Superdex peptide, Superdex 30, Superdex 200, Superose 6, Superose 12, Sephacryl, Sphadex (all from Amersham Biosciences, Vienna, Austria), Biogel P, Agarose-gel, Fracto-gel or Ultro-gel. A most preferred gel filtration system, also employed in the appended examples, is a HiLoad 16/60 Superdex 75 gel filtration column.

Protein precipitation techniques comprise, inter alia, Dextran sulphate-, Polyethylene glycol (PEG) 4000-8000-, Acetone-, Protamne sulphate-, Streptomycin sulphate-, pH-shift-precipitations. Preferably, said protein precipitation is carried out by ammonium sulfate precipitation. More preferably a 30%, most preferably a 38% saturated (NH₄)SO₄-solution is employed.

Accordingly, an example of such a purification method is a three step purification comprising: 1. protein precipitation, 2. ion exchange chromatography 3. gel filtration. As illustrated in appended example 2, which comprises more details, this precipitation method may preferably be carried out by an ammonium sulfate precipitation using 30% saturated (NH₄)₂SO₄ solution, most preferably a 38% saturated (NH₄)SO₄-solution, an ion exchange chromatography using a Mono Q HR10/10 column (Amersham Biosciences, Vienna, Austria) and a gel filtration using a HiLoad 16/60 Superdex 75 gel filtration column (Amersham Biosciences, Vienna, Austria) It is also envisaged that, as step before the protein precipitation or in addition to the protein precipitation, the crude cell lysate is centrifuged, for example at 70 000×g for 1 hour.

As mentioned herein above, the antibodies obtained after eliciting an immune response against the highly purified HPV-16 E7 (preferably native, highly purified HPV-16 E7) or a fragment thereof are further purified, in particular affinity purified. Preferably, said affinity purification of the obtained antibodies is carried out over immobilized HPV-16 E7 protein or a fragment thereof. Most preferably, said HPV-16 E7 protein or a fragment thereof is immobilized on PVDF membranes, nitrocellulose, sepharose, agarose, DEAE-cellulose or DEAE. As illustrated in the appended examples, one possibility of affinity purifying the HPV-16 E7 or a fragment thereof comprises the immobilization of HPV-16 E7 or said fragment on PVDF membranes. Immobilized HPV-16 E7 protein is incubated with the polyclonal HPV-16 E7 antiserum, washed, and the affinity purified antibodies are eluted by an acid gradient from the immobilized HPV-16 E7 protein. Corresponding protocols are illustrated in the appended examples.

The elution of bound anti-HPV-16 E7 antibodies may be carried out by methods known in the art which, inter alia, comprise acid gradients or salt gradients.

In a particularly preferred embodiment, the HPV-16 E7 protein is prepared as described in Examples 1 and 2, appended hereto.

Most preferably, the “non-human vertebrate” mentioned herein above is selected from the group consisting of rat, mouse, rabbit, chicken, sheep, horse, goat, pig and donkey. Most preferably said vertebrate is a chinchilla bastard rabbit or goat.

The antibodies of the present invention provide for the first time a reliable tool in the (immuno)-histochemical detection of an HPV-16 E7 infection and/or in cancer diagnostic. The antibodies provided herein are, inter alia, useful in the direct measurement of expressed E7 oncoprotein in biological samples, for example in Pap-smears, samples from cervix biopsies, low or high grade squamous, intraepithelial lesions, in samples from prostate biopsies, in particular from fine needle aspiration biopsies.

It was surprisingly found that the anti-HPV-16 E7 antibodies produced according to the above-described method are, in contrast to antibodies of the prior art, capable of reliably detecting expressed E7 in a variety of biological samples. It is of particular note that the antibodies of the invention are also capable of detecting E7 in fixed material, e.g. in formaldehyde-fixed biological samples. The detection is also possible in paraffin- or frozen sections of biological samples and tissue. As documented in the appended examples, the described antibodies may be employed in (immuno)-histological techniques, like immunostainings of biological tissue (e.g. cervix tissue) or in probes derived from fine needle aspiration biopsies (e.g. prostate tissue).

Accordingly, the present invention provides for improved diagnostic tools for the detection of an HPV-16 infection. The antibodies of the present invention are in particular useful for the detection of HPV-16 E7 in Pap-smears.

The detection of, e.g., enhanced E7 oncoprotein expression level by the provided antibody(ies) allows to identify pre-neoplastic lesions with a particularly high risk for malignant progression and invasive cancers on histological probes and/or in cytological smears. This helps to improve current limitations in cancer screening, diagnosis, and therapy control, in particular in cervical and prostate cancer. The described antibodies provide for useful tools in the classification of sexually transmitted diseases or of cancer. Furthermore, these antibodies against highly purified HPV-16 E7 protein or a fragment thereof recognise the HPV-16 E7 oncoprotein in neoplastic cells derived from, e.g., cervical smears, in paraffin- or in frozen-sections from biopsies of patients. Thus, these antibodies have major diagnostic potential as markers of malignant transformation in, inter alia, carcinogenesis, e.g. cervical carcinogenesis or prostate carcinogenesis.

The antibodies described herein have major advantages over the antibodies of the prior art, e.g. commercially available antibodies as, inter alia, provided by Santa Cruz Biotechnologies or Zymed Laboratories. In contrast to antibodies and antibody-reagents provided by the prior art, the antibody/antibodies/sera described herein are highly specific and do not provide for high number of “false-positive” signals, i.e. of a “positive” immunobiochemical signal in samples or cells which are HPV-16 negative or which do not express the HPV-16 E7 protein or a fragment thereof. Furthermore, the herein described antibodies are not only highly specific but do also not provide for a high number of “false-negative” immunobiochemical signals. As illustrated in the appended examples the antibodies of the invention may be, inter alia, tested for this reliability in transfection studies. For example, cultured cells, preferably human U2-OS cells may be transfected with a vector heterologously expressing E7, for example a vector which provides for CMV-driven expression of HPV-16 E7. As a negative control, further U2-OS-cells may be transfected with an expression vector which does not express said E7 protein. “False positive” signals are evaluated by the amount of cells which are not transfected with the E7-expressing vector, but which, nevertheless, give a positive signal in immunobiological screenings, e.g. immunofluorescence microscopy. Preferably, less than 15%, more preferably less than 10%, even more preferably less than 5%, most preferably none of the cultured cells which do not (transciently or permanently) express E7-proteins (“negative control cells”) are stained by the antibody described herein. “False negative” signals are evaluated by the amount of cells which are positively transfected with the E7-expressing vector or which are positive infected by E7-expressing HPV-16, but which give a negative signal in immunobiological screenings, e.g. immunofluorescence microscopy.

The invention also provides for the use of an anti-HPV-16 E7 antibody of the invention for the preparation of a diagnostic composition for the (immuno-)histological detection of expressed HPV-16 E7 in a biological sample. Preferably, said (immuno-)histological detection is carried out on Pap-smears (cervical smears), cervical (carcinoma) biopsies or prostate biopsies, like fine needle aspiration biopsies. It is also envisaged that said (immuno)histological detection is carried out on smears and/or biopsies of anogenital dysplasias. Such dysplasias may lead to, inter alia, anal squamous intraepithelial lesions and neoplasias (ASIL, AIN) or anal, penile and reproductive tract cancers. In this context, also HPV diagnostic, in particular HPV-16 diagnostic is envisaged which comprises the analysis of samples derived from men, belonging to risk groups of sexually transmittable diseases, like bisexual and homosexual men. Yet, the diagnostic compositions described herein are useful in diagnostic settings of both, men and women, and independently from their sexual orientation. Also envisaged is the use of the inventive anti-HPV-16 E7 antibody and the diagnostic composition described herein in the detection of expressed HPV-16 E7 in smears and biopsies of head and neck tissue, mamma tissue, prostate tissues, penile tissue, cervix tissue and the like.

Most preferably said diagnostic composition is used for evaluating the acquisition of a sexually transmitted disease or the risk of developing cancer, for measuring the status of an existing sexually transmitted disease or cancer, or for screening the therapy efficiency in the treatment of a sexually transmitted disease or cancer.

Furthermore, the invention relates to a method for the preparation of a diagnostic composition comprising the step of formulating the inventive anti-HPV-16 E7 with a diagnostically acceptable carrier, diluent, buffer, or storage solution. It is also envisaged that in the use or the method of the present invention, said diagnostic composition further comprises suitable means for detection, for example secondary labelled antibodies or fragments thereof.

A variety of techniques are available for labeling biomolecules, are well known to the person skilled in the art and are considered to be within the scope of the present invention. Such techniques are, e.g., described in Tijssen, “Practice and theory of enzyme immuno assays”, Burden, RH and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc.

There are many different labels and methods of labeling known to those of ordinary skill in the art. Examples of the types of labels which can be used in the present invention include enzymes, radioisotopes, colloidal metals, fluorescent compounds, chemiluminescent compounds, and bioluminescent compounds. Preferred are labels to be detected in immunohistochemical techniques.

Commonly used labels comprise, inter alia, fluorochromes (like fluorescein, rhodamine, Texas Red, Cy3, Cy5, etc.), enzymes (like, peroxidase, horse radish peroxidase, β-galactosidase, alkaline phosphatase), radioactive isotopes (like ³²P or ¹²⁵I), biotin, digoxygenin, colloidal metals, chemi- or bioluminescent compounds (like dioxetanes, luminol or acridiniums). Labeling procedures, like covalent coupling of enzymes or biotinyl groups, iodinations, phosphorylations, biotinylations, etc. are well known in the art. It is of note that the antibodies of the invention may also be detected by secondary methods, like indirect immuno-fluorescence. Accordingly, detectably labeled secondary antibodies may be employed in the methods and uses of the present invention.

As mentioned above, direct and indirect detection methods comprise, but are not limited to, fluorescence microscopy, direct and indirect enzymatic reactions and the detection by microscopic means as well as direct detection by eye-visible signals resulting, inter alia, from accumulation of dye-labeled antibodies or the secondary detection of antibodies. Similarly, as detailed below, the detection of E7 protein by the inventive antibodies may comprise the detection of soluble or solubilized E7 protein in fluid samples or solubilized samples. Such methods preferably comprise, inter alia, ELISA-, FIA-, CLIA- or RIA-tests (see also below), or the use of test sticks as described below. Commonly used detection assays comprise, accordingly, radioisotopic or non-radioisotopic methods. These comprise, inter alia, Westernblotting, overlay-assays, RIA (Radioimmuno Assay) and IRMA (Immune Radioimmunometric Assay), EIA (Enzyme Immuno Assay), ELISA (Enzyme Linked Immuno Sorbent Assay), FIA (Fluorescent Immuno Assay), CLIA (Chemioluminescent Immune Assay), lateral flow immunoassay, as well as the use of test sticks detailed herein.

Accordingly, the invention also provides for a diagnostic composition comprising the anti-HPV-16 E7 antibody of the invention or obtained by the method of the invention

Said diagnostic composition may comprise the antibody molecules of the present invention, in soluble form/liquid phase but it is also envisaged that said antibodies are bound to/attached to and/or linked to a solid support. Said diagnostic composition may be employed in samples derived from solid tissue as well as in samples which comprise fluid probes. These fluid samples may be selected, inter alia, from blood, serum, plasma, sputum, urine, ejaculate, sperm. It is also envisaged and described herein that solid samples/probes are solubilized be/are they are tested with the diagnostic composition of the present invention. Yet, in a most preferred embodiment, the antibodies/sera of the present invention (and therefore the diagnostic composition) is used on smears, like Pap-smears.

Solid supports may be used in combination with the diagnostic compostion as defined herein or the antibodies, antibody fragments or antibody derivatives of the present invention may be directly bound to said solid supports. Such supports are well known in the art and comprise, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The antibodies of the present invention may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. Appropriate labels and methods for labeling have been identified above. In a preferred embodiment said diagnostic composition comprises the use of immobilized inventive antibodies.

In accordance with this invention, cost-efficient, rapid and reliable diagnostic tests and test kits may be developed. For example, a teststick may be produced that is capable to indicate HPV induced tumor development in cell lysates of cervical smears. Such lysates are often taken from material from cervix uteri, which are routinely lysed in sample buffers. Yet, the test kits of the invention may also be employed in tests for HPV-16 E7 in other samples, like (blood) serum or lysates from further biopsies or smears, like analogenital biopsies or smears. Such a test, comprising the use of test sticks or other solid matrices, is established on the principle of a ‘lateral flow system’. It is within the skill of a person skilled in the art to develop tests/test kits or means for testing which comprise, inter alia, the preparation of a test stick directly or indirectly conjugated with the antibodies of the invention. One, non-limititing example may be the preparation a “cassette housing” with windows for sample application and optical evaluation of results (comprising test and control lines, respectively) whereby said “housing” comprises a support backing as a carrier for an analytical membrane, a sample application pad, a conjugate release pad and an absorbent pad. The conjugate release pad may be prepared with substrates, comprising (conjugated) anti-E7 antibody of this invention, whereby said conjugation may, inter alia, be gold- or latex conjugation. The analytical membrane area in the test window may, inter alia, be prepared with different reagents in separated lines fixed to said membrane. It is envisaged that the testline carries the inventive anti-E7 antibody and the control lines may comprise E7 protein as well as (an) secondary antibody antibodies, like (an) anti-rabbit (or anti-goat or the like) antibody or antibodies. Furthermore, a/the control line may comprise other detections means for further/other sample compounds. In the test illustrated here, the function of the control lines is to monitor the efficiency of the test/teststick and the conjugated antibodies and to exclude false positive and negative results by interfering substances. Similar assays and test means are known in the art and comprise, inter alia, pregnancy tests based on specific antibody-antigen interactions. The test stick described herein may not only be employed in cell lysates of tissue(s) to be tested but also in body fluids, like blood, serum, plasma, sputum, urine, ejaculate, sperm and the like.

In a further embodiment the invention provides for a kit comprising an anti-HPV-16 E7 antibody of the invention or a diagnostic composition of the invention.

Advantageously, the kit of the present invention further comprises, optionally (a) buffer(s), storage solutions and/or remaining reagents or materials required for the conduct of medical, scientific or diagnostic assays and purposes. Furthermore, parts of the kit of the invention can be packaged individually in vials or bottles or in combination in containers or multicontainer units. The kit may also comprise an instruction sheet to carry out the (diagnostic) methods of the present invention.

The kit of the present invention may be advantageously used, inter alia, for carrying out the (diagnostic) methods of the invention and could be employed in a variety of applications referred herein, e.g., as diagnostic kits, as research tools or medical tools. Additionally, the kit of the invention may contain means for detection suitable for scientific, medical and/or diagnostic purposes, like e.g. secondary antibodies as described above. The manufacture of the kits follows preferably standard procedures which are known to the person skilled in the art.

In another aspect the present invention relates to an in vitro method for the detection of a sexually transmittable disease or cancer comprising the steps of

(a) incubating a biological sample with anti-HPV-16 E7 antibodies of the invention; and

(b) measuring and/or detecting specifically-bound anti-HPV-16 E7 antibodies whereby the presence, the absence or the amount of specifically-bound anti-HPV-16 E7 antibodies is indicative for said sexually transmittable disease or cancer. It is further preferred that this in vitro method comprises a further step (c), whereby in said step (c) the presence, the absence or the amount of specifically-bound anti-HPV-16 E7 antibodies of step (b) is compared to the presence, the absence or the amount of specifically-bound anti-HPV-16 E7 antibodies in a negative or a positive control sample or in both control samples.

The measurement and/or detection of specifically “bound anti-HPV-16 E7 antibodies” may be carried out as described above, for example by the detection of directly or indirectly labelled, bound antibody molecules of the invention. Said measuring and detection methods may also comprise automated and/or computer-controlled detection methods.

Such in vitro methods of the invention are also illustrative in the appended examples and may be, inter alia, employed to detect the presence or absence of an HPV16 infection, to evaluate whether a HPV16 infection is merely transient or an asymptomatic HPV16 infection. It is of note that the antibodies of the present invention may be employed in the above described method in order to evaluate the absence or presence of a proliferative disorder, like, e.g. cervix carcinoma, prostata carcinoma, breast cancer, anogenital cancer, penile cancer and head and neck cancer. Furthermore, the antibodies may be employed to evaluate the class of a proliferative disorder, for example it can be evaluated whether a prostatic carcinoma is HPV16 dependent or independent. It is, e.g., envisaged that patients whose serum comprises prostatic-specific antigen (PSA) or who have a positive result in fine-needle aspiration biopsies of prostatic tissue are further examined for the presence or absence of HPV-16 E7, employing the antibodies of the invention and methods disclosed herein. Such a diagnostic method allows for the distinction of HPV16-positive and HPV16-negative prostate carcinomas and the medical intervention may be chosen accordingly.

As mentioned above, the biological sample is preferably a cervix or a prostatic sample, most preferably a Pap-smear or a fine needle aspiration biopsy.

In contrast to previous technology, namely the detection of HPV-16 DNA in prostate cancer biopsies, the detection of high-level HPV-16 E7 expression in prostate cancer samples allows the conclusion that in these samples the E7 oncoprotein, which is the major transforming protein of the virus, is actively expressed. The person skilled in the art knows that expression of the E7 oncoprotein in any cell results in the inactivation of several important tumor suppressor mechanisms, as reviewed in Zwerschke (2000, Adv. Cancer Res. 78, 1-29). This indicates that there is a high risk for malignant progression of this lesion. It has to be stressed that currently physicians do not consider HPV-dependent malignant progression of prostate cancers, since detection of HPV oncoproteins in prostate cancer specimens was not possible with the techniques of the prior art. The antibodies described herein allow significant progress in clinical research. Furthermore, it is anticipated that with the advent of specific antiviral drugs and/or treatments directed against high-risk papillomaviruses, the detection of HPV-16 E7 in prostate cancer specimens will direct the physician to new modes of treatment for this important malignancy.

In accordance with this invention, the biological sample to be tested and/or evaluated with the inventive anti-HPV-16 E7 antibody may be a solid sample as well as a soluble/solubilized sample. Even if one of the most preferred uses of the inventive antibody/antibodies comprises the diagnostic use in immunohistochemical assays, in particular on smears, further methods of diagnosis employing the inventive antibodies are envisaged in this invention. These further methods are described and illustrated herein and comprise the use of solid and non-solid phase immunoassays, like ELISA-, RIA-tests or the use of (antibody-covered) tests sticks, magnetic or polystyrol beads and the like.

Besides samples derived from cervix, anogenital tissue head- and neck tissue and/or prostatic tissue, it is also envisaged that the inventive anti-HPV-16 E7 antibody is employed in diagnostic samples derived from mamma/mamma tissue. The antibody of the present invention is particularly useful in screening of mamma tissue obtained from patients who suffer or had suffered from a cervix carcinoma and may develop, e.g. due to metastasis, a mamma carcinoma. Accordingly, the present invention also relates to an in vitro method for detection of a mamma/breast cancer, in particular of a mamma cancer in a patient who suffers or who has suffered from, in particular a cervix carcinoma/cervical cancer. Said in vitro method comprises the incubation of mamma tissue (solid or solubilized) with anti-HPV-16 E7 antibodies of the invention and the measurement and/or detection of specifically-bound anti-HPV-16 E7 antibodies, whereby the presence, the absence or the amount of specifically bound anti-HPV-16 E7 antibody is indicative for mamma/breast cancer. In particular, a positive signal of specifically bound E7 antibody of the present invention is indicative for a mamma carcinoma/breast cancer, in particular a mamma carcinoma being a secondary tumor or a metastasis from a primary tumor, like a cervix carcinoma or an anogenital cancer.

The invention, accordingly, provides for the use of an anti-HPV-16 E7 antibody, a diagnostic composition or a kit of the invention in an in vitro method for the detection of a sexually transmittable disease or cancer. Said sexually transmitted disease is, preferably an HPV16-infection or said cancer is cervical cancer, breast cancer, prostate cancer, anogenital cancer/anogenital neoplasia (AIN), penil cancer or head and neck cancer. The feasibility of a successful HPV-diagnostic, in particular HPV-16 E7 diagnostic on smears is described in the appended examples.

In another embodiment, the present invention provides for a method for production of an anti-HPV-16 E7 antibody comprising the steps of

(a) eliciting an in vivo humoral response against highly purified, HPV-16 E7 protein or a fragment thereof in a non-human vertebrate; and

(b) affinity-purifying antibodies as obtained in the eliciting-step (a).

In a most preferred embodiment, the highly purified HPV-16 E7 proteins or a fragment thereof to be used in the immunization protocol described herein and illustrated in the appended examples is a native, highly purified HPV-16 E7 protein or a fragment thereof. The term “native” as used in accordance with this invention is explained herein above and illustrated in the appended examples. With respect to the preferred embodiments the same applies, mutatis mutandis, as described herein above for the anti-HPV-16 E7 antibody.

The Figures show:

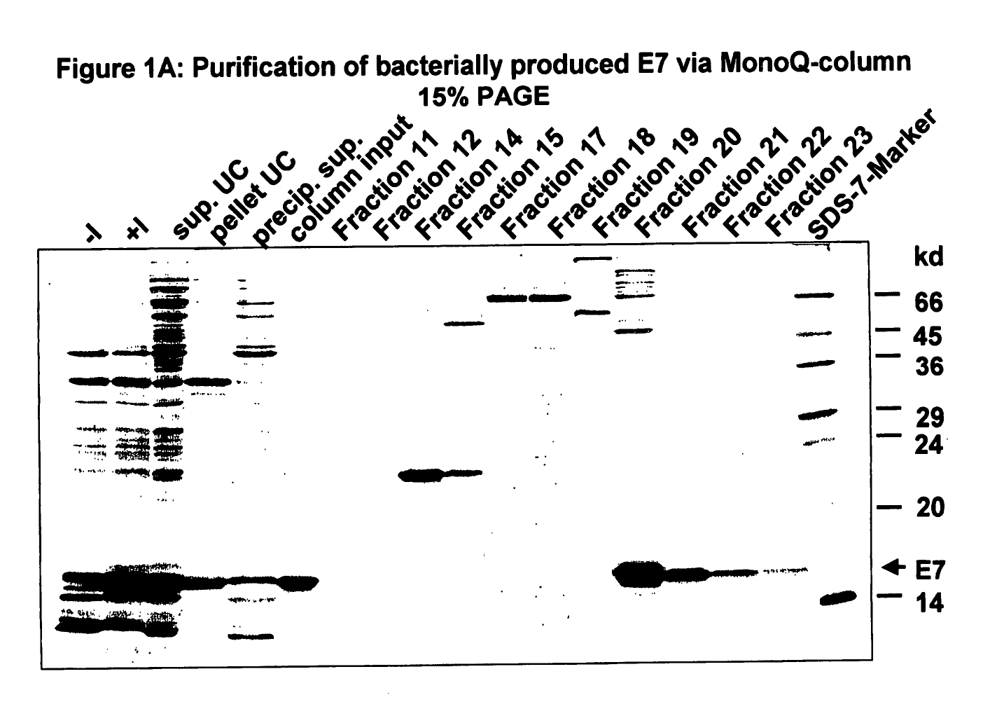

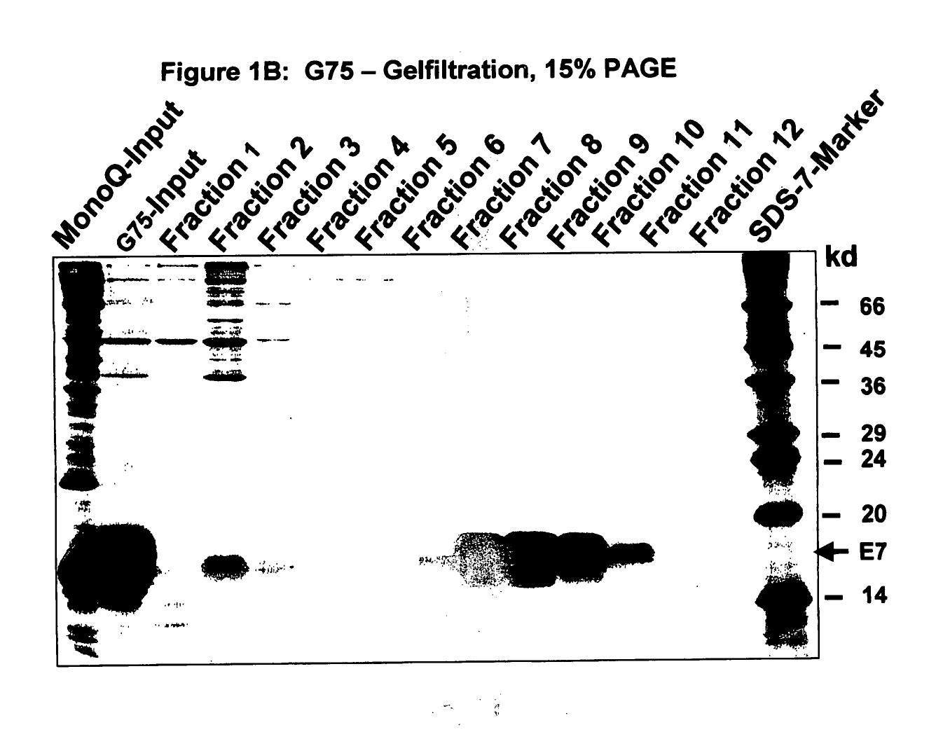

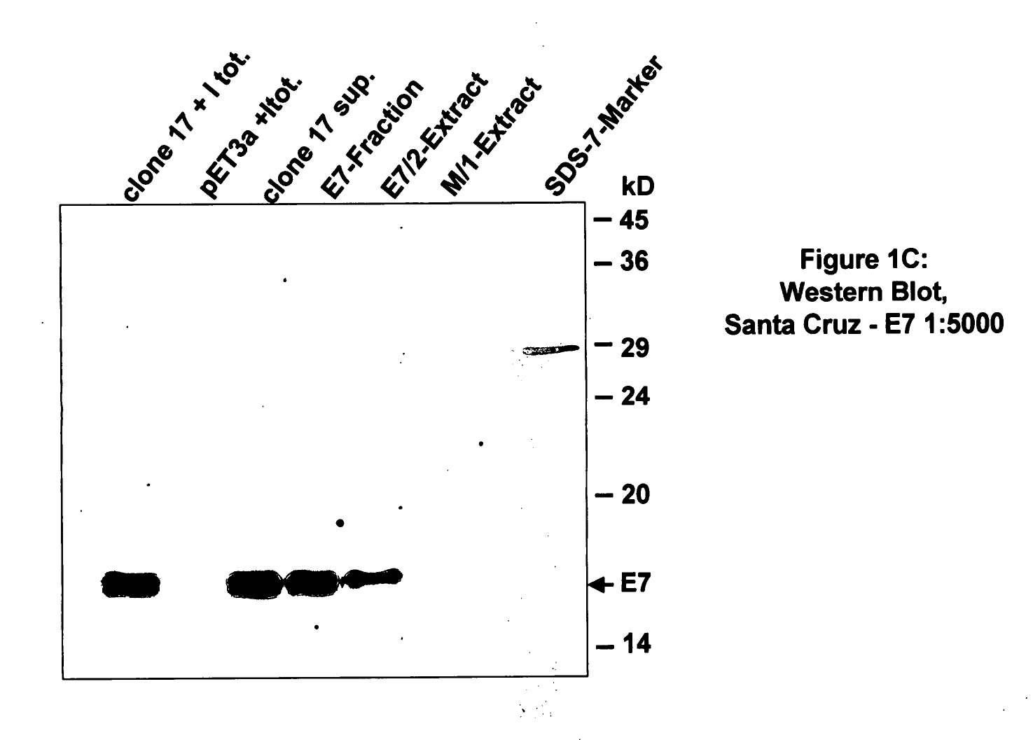

FIG. 1: Purification of the HPV-16 E7 oncoprotein. Bacterial expressed recombinant HPV-16 E7 was stepwise purified by ammonium sulfate precipitation, anion-exchange chromatography on MonoQ and gelfiltration on a Sephadex G75 column. (A, B) Samples were separated by gel electrophoresis, and purification was documented by coomassie staining of the fractions as indicated. Purity of the HPV-16 E7 protein was confirmed by Western blotting using a monoclonal anti E7 antibody (Santa Cruz, Vienna, Austria) (C).

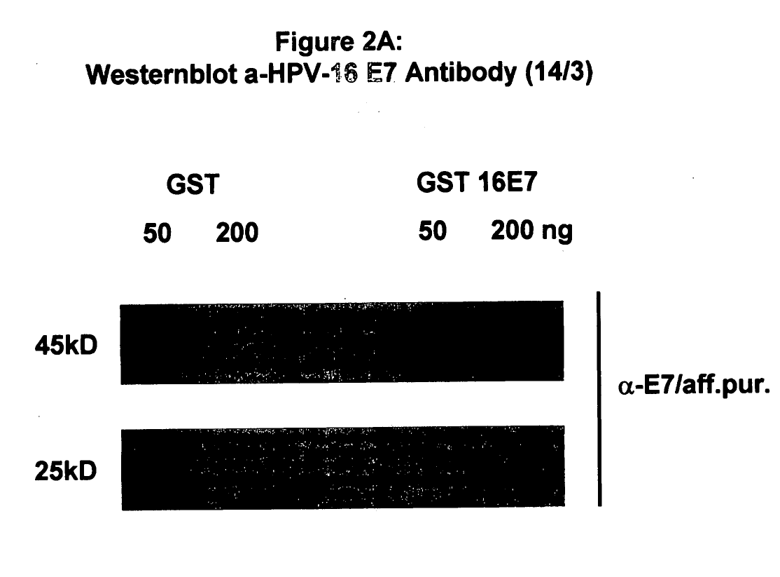

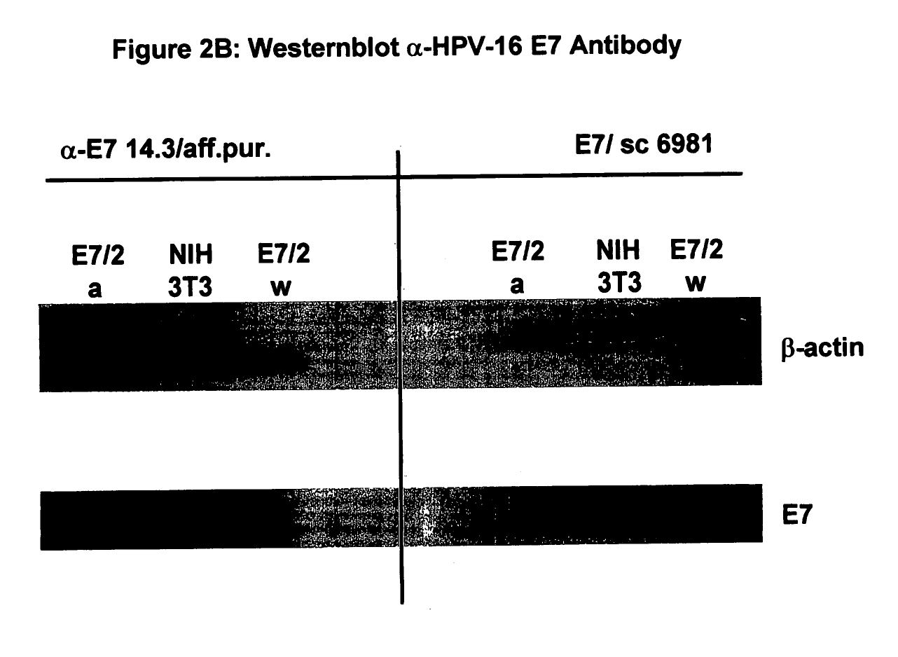

FIG. 2: Test of the affinity purified anti-HPV-16 E7 antibodies (14/3) in westernblot analysis. A. Purified GST and GST-HPV-16 E7 proteins were separated by SDS-polyacrylamide gel electrophoresis, and the GST-HPV-16 E7 protein was detected by westernblotting. B. The HPV-16 E7 expressing cells E7/2 and the control cells were subjected to lysis. Subsequently lysates were separated by SDS-polyacrylamide gel electrophoresis and probed with antibodies to HPV-16 E7 and beta actin (input control), as indicated.

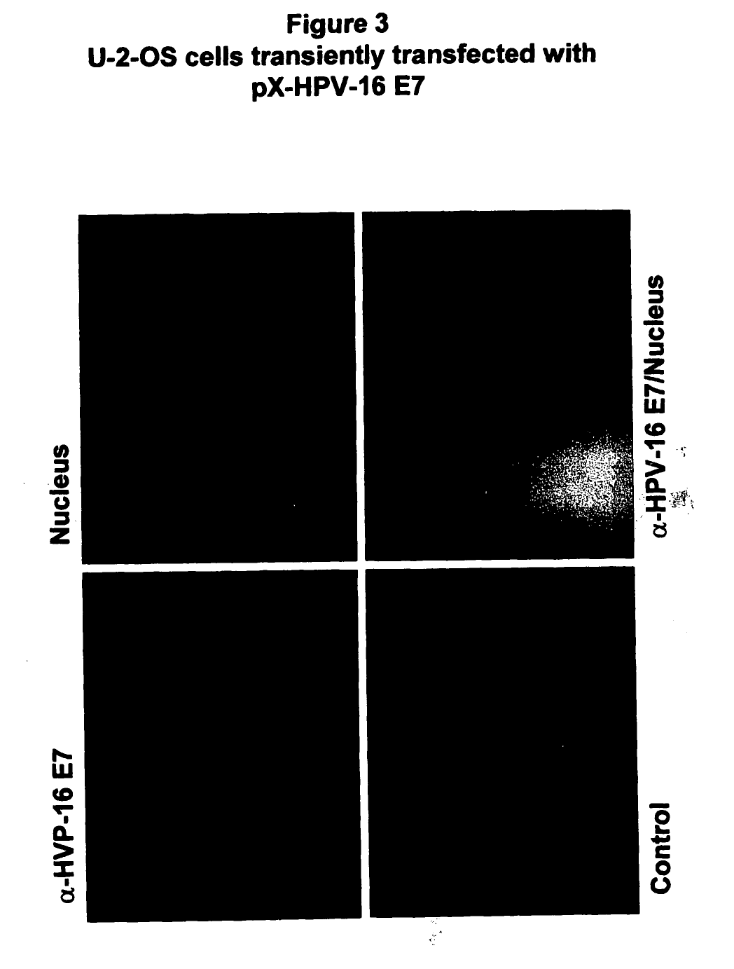

FIG. 3: Detection of HPV-16 E7 after transient expression in human cells. U-2OS cells were transiently transfected with expression vectors for HPV-16 E7, as indicated. At 26 h post transfection, cells were processed for indirect immunofluorescence microscopy and viewed by using a confocal scanning system. Cells were stained with anti-E7 antibodies clone 14/3 (α-HPV-16 E7), preimmune serum (control), TroPro3 (nucleus) or both anti-E7 antibodies and TroPro3 (α-HPV-16 E7/nucleus), as indicated.

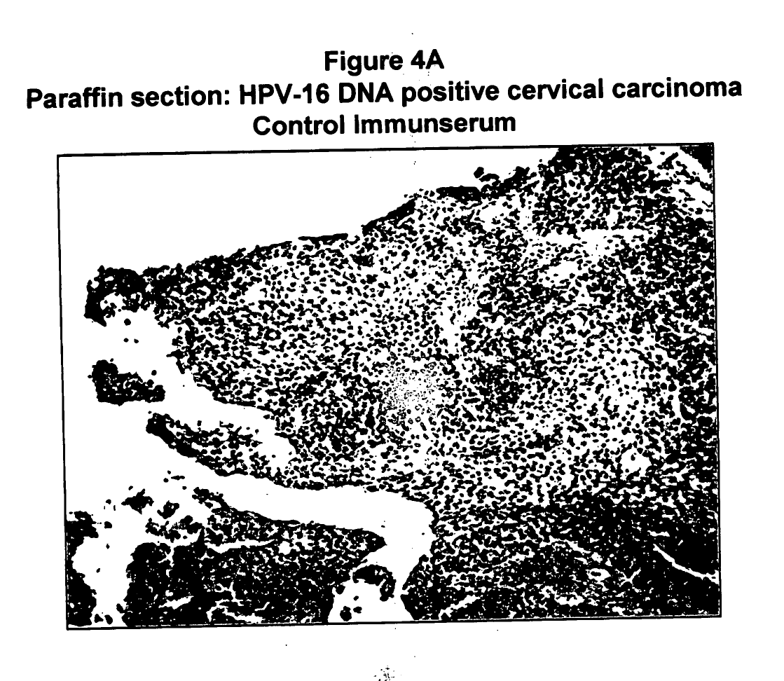

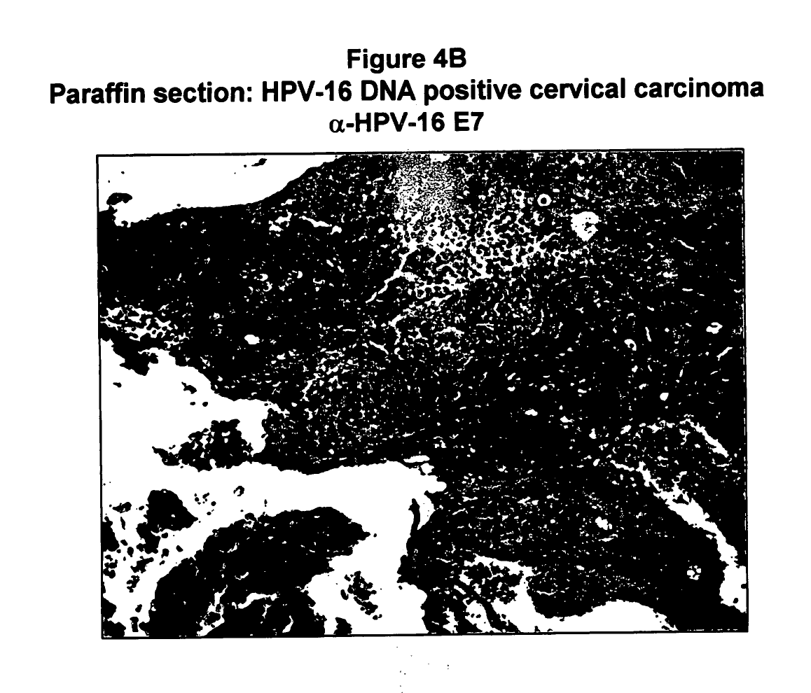

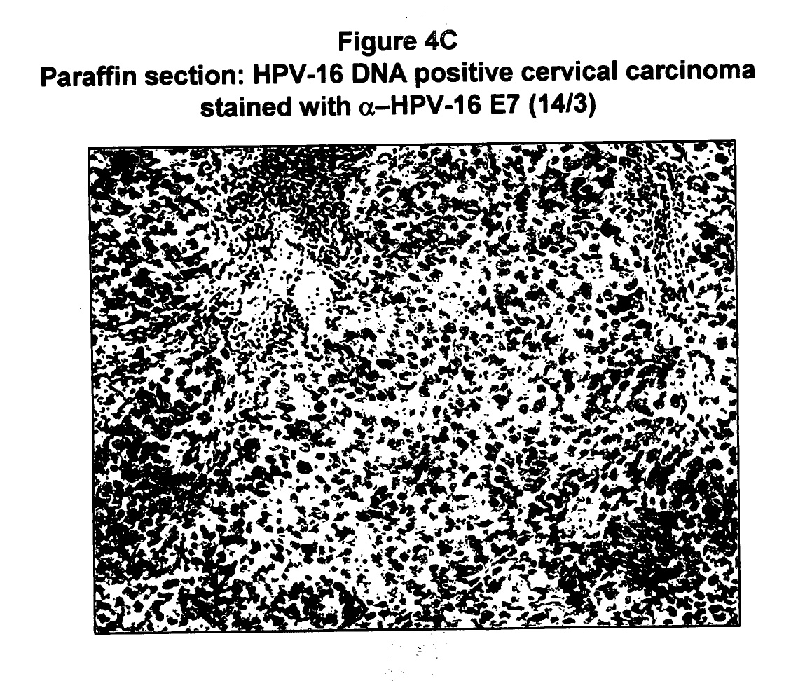

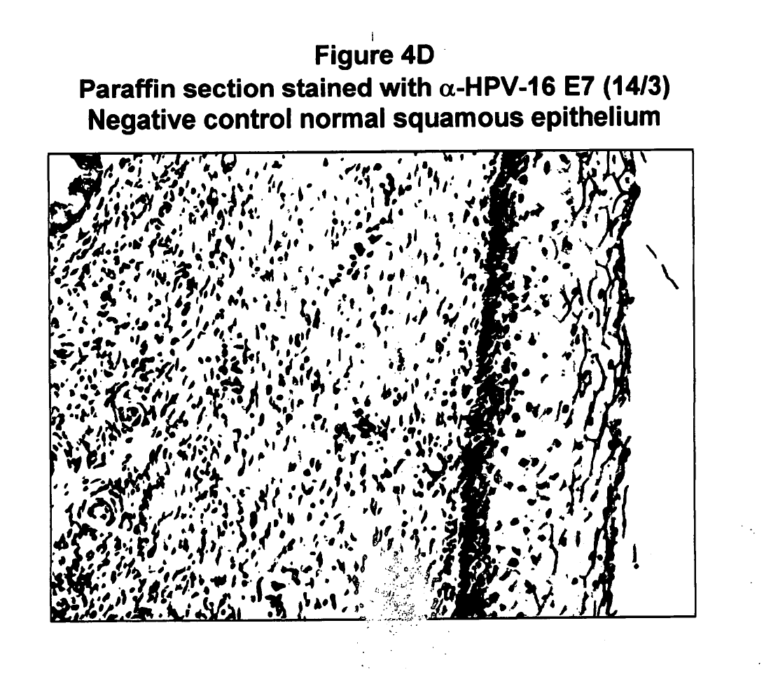

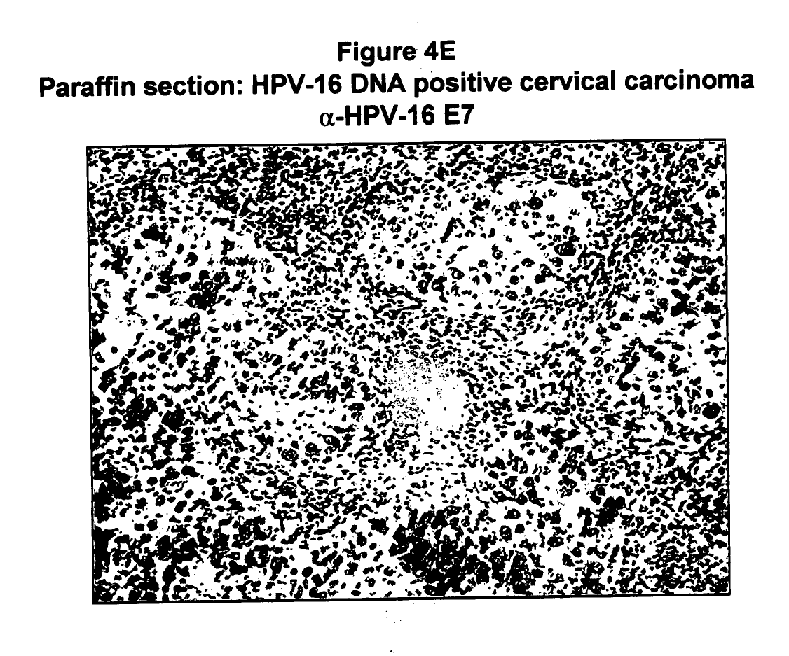

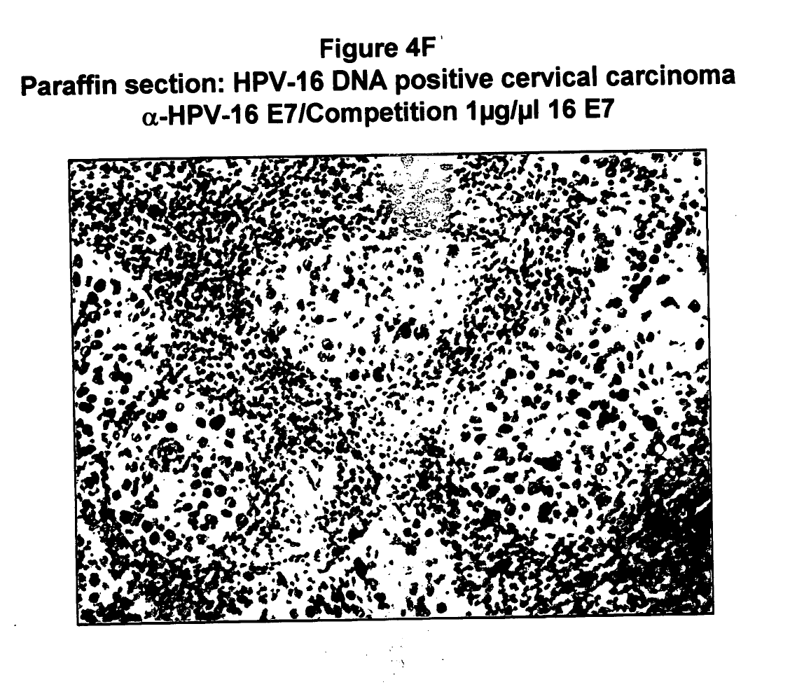

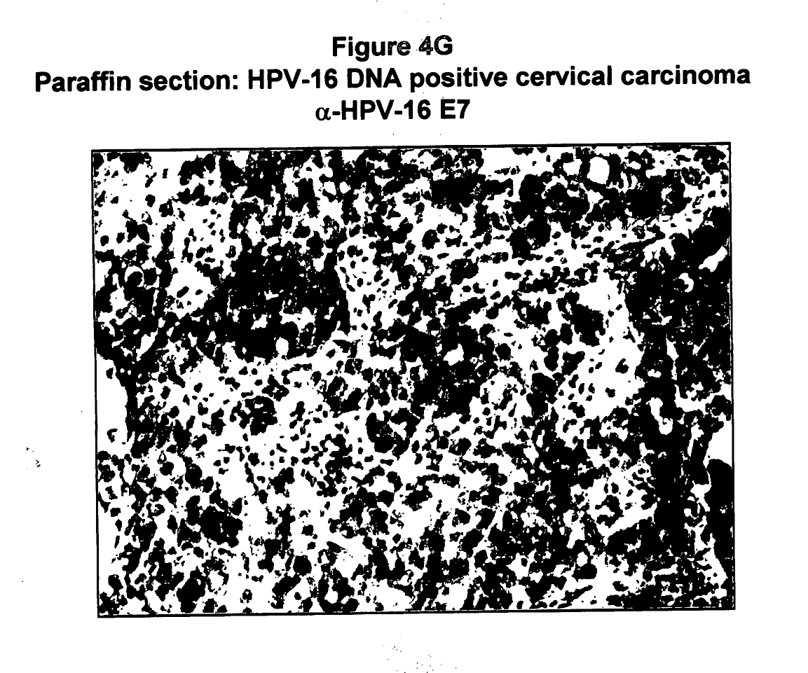

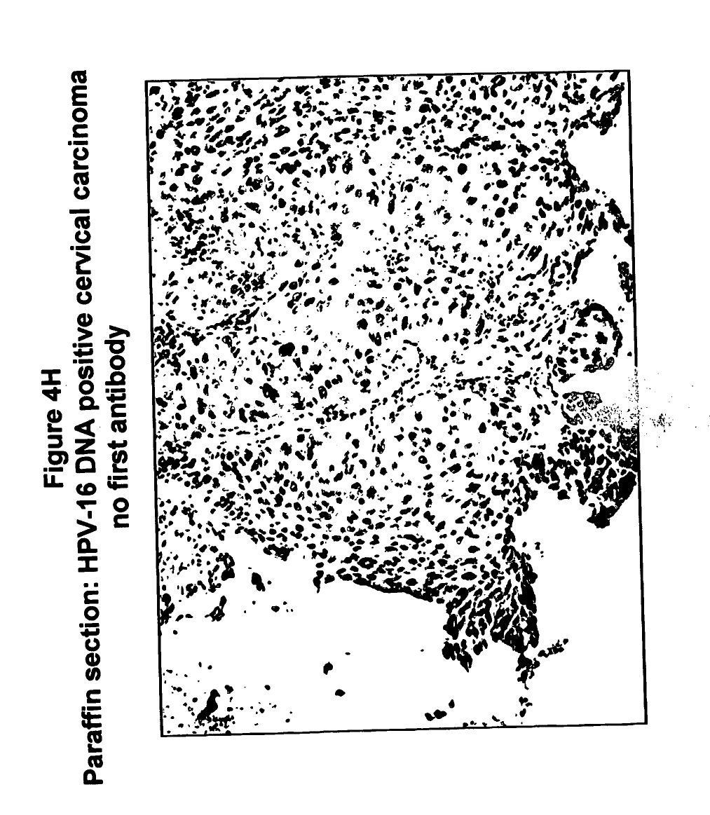

FIG. 4: Immunoperoxidase staining of paraffin sections of normal cervix and cervical carcinomas with affinity purified polyclonal antibodies against HPV-16 E7. Paraffin sections of normal cervix and a cervical carcinoma were immunostained for HPV-16 E7 by the immunoperoxidase method as described in material and methods. (A) In cervical carcinoma tissue, epithelial cells are negative with the preimmunserum. (B, C, E, G) In cervical carcinoma tissues anti-HPV-16 E7 antibodies stain virtually all cells in the tumor islets. (D) In normal cervical tissue, epithelial cells are negative with these antibodies. (F) In cervical carcinoma tissues staining by the anti-HPV-16 E7 antibodies can be competed out by preincubation of the antibodies with purified HPV-16 E7 antigen. (H) Control, no staining of cervical carcinoma tissues was obtained by adding only the horse radish conjugated secondary anti rabbit IgG.

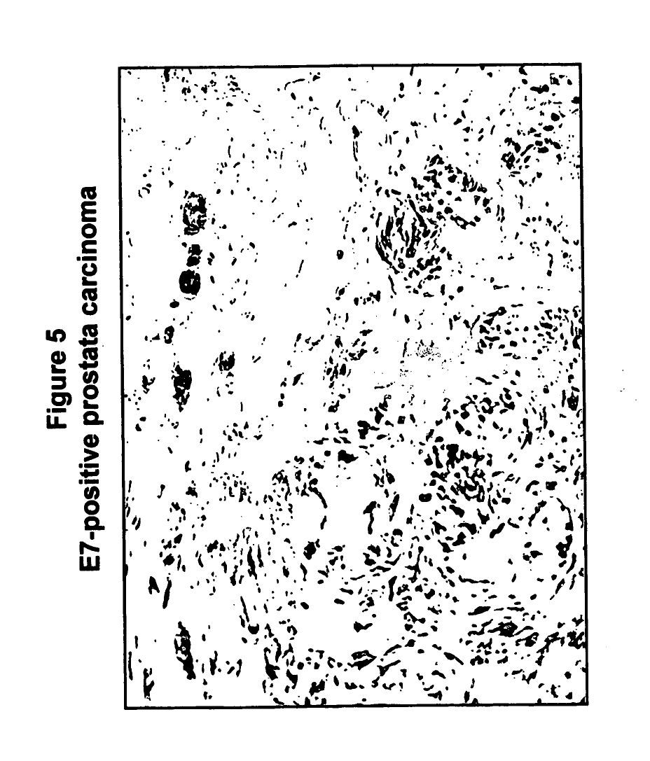

FIG. 5: Immunoperoxidase staining of cells obtained from prostate carcinoma patients. Biopsies were taken from 60 prostate carcinoma patients and samples from 60 patients were applied to an object slide together with negative controls. These slides are known to the expert as “tissue microarrays” (Skacel, 2002, Appl. Immunohistochem. Mol. Morphol. 10, 1-6). Tissue micorarrays were stained with antibodies to HPV-16 E7 as described in FIG. 4 for cervical biopsies. In this experiment, a subset of the carcinoma biopsies stained positive for HPV-16 E7, whereas other biopsies from different prostate cancer patients stained negative.

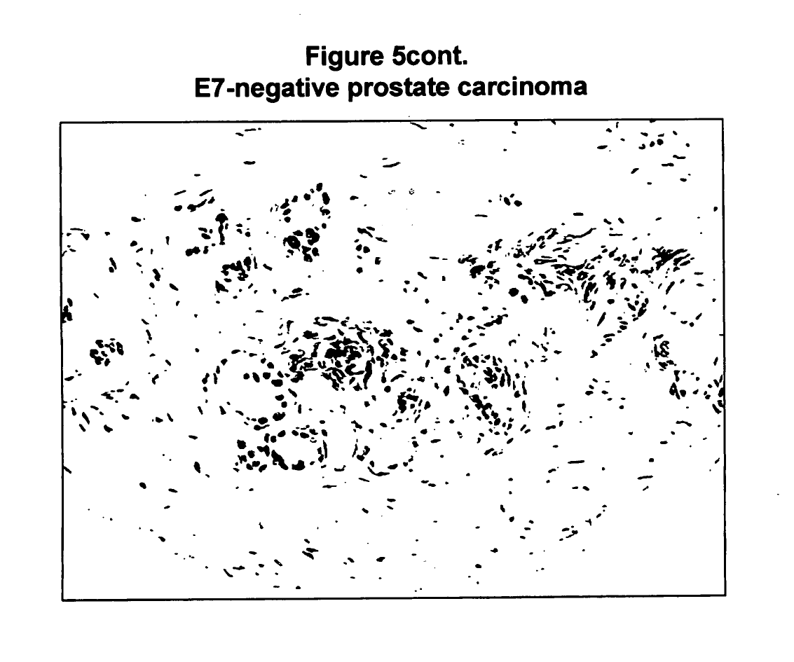

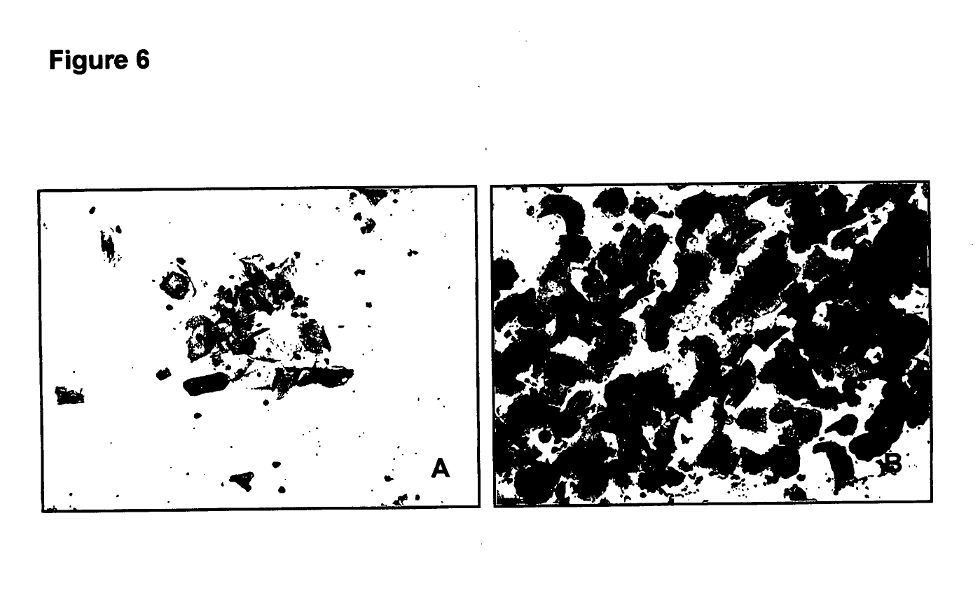

FIG. 6: Cells from surface layers of the ectocervical epithelium were spread out on glass object slides and immunoperoxidase stained by the anti-HPV-16 E7 antibodies (brown). The cells were counterstained with Hemalaun (grey/blue) and viewed by brightfield microscopy. The HPV-DNA status of the specimens was analyzed by PCR. (A) No brown staining was observed in cells from normal (Pap II) HPV-DNA negative ectocervical smear. (B) Cells from HPV-16 DNA positive cytological abnormal (Pap IIID) ectocervical smear were stained brown by the antibodies.

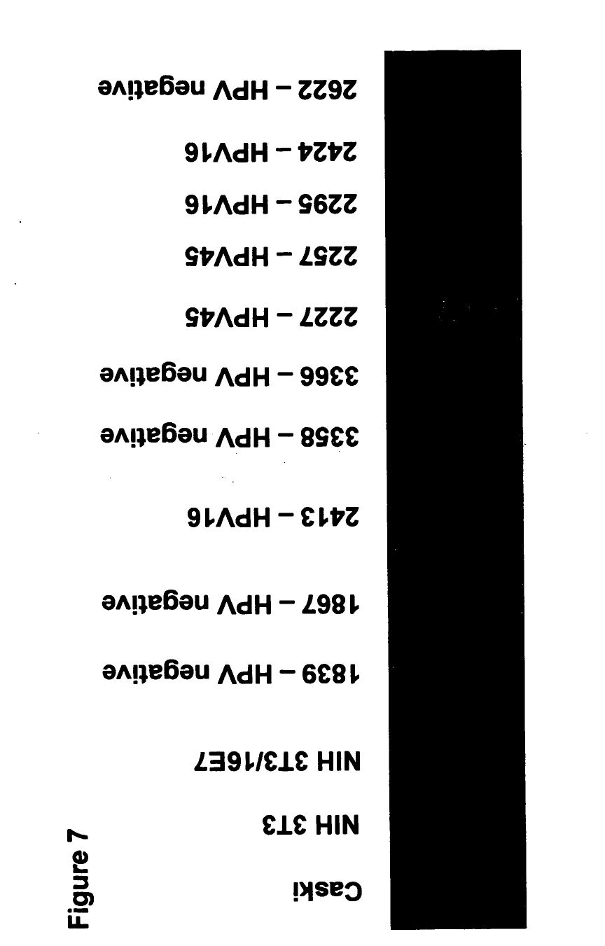

FIG. 7: Expression of the HPV-16 E7 oncoprotein in biopsies derived from cervical carcinoma patients. Three HPV-16 positive cervical carcinomas and seven HPV-16 negative cervical tissues were analysed for the expression of the HPV-16 E7 protein. Lysates, 0.5 mg each, were separated by SDS-polyacrylamide gel electrophoresis, and the HPV-16 E7 protein was detected by Western blotting. As controls, lysates from CaSki cells, an established cervical carcinoma cell line (obtained from DKFZ Heidelberg, Germany), NIH3T3 mouse fibroblasts and NIH3T3/16E7 cells, a cell line derived from NIH3T3 cells by stable transfection with the pMOHPV16E/ expression vector (Edmonds (1989), J. Virol. 63, 2650-2656 ), were assayed.

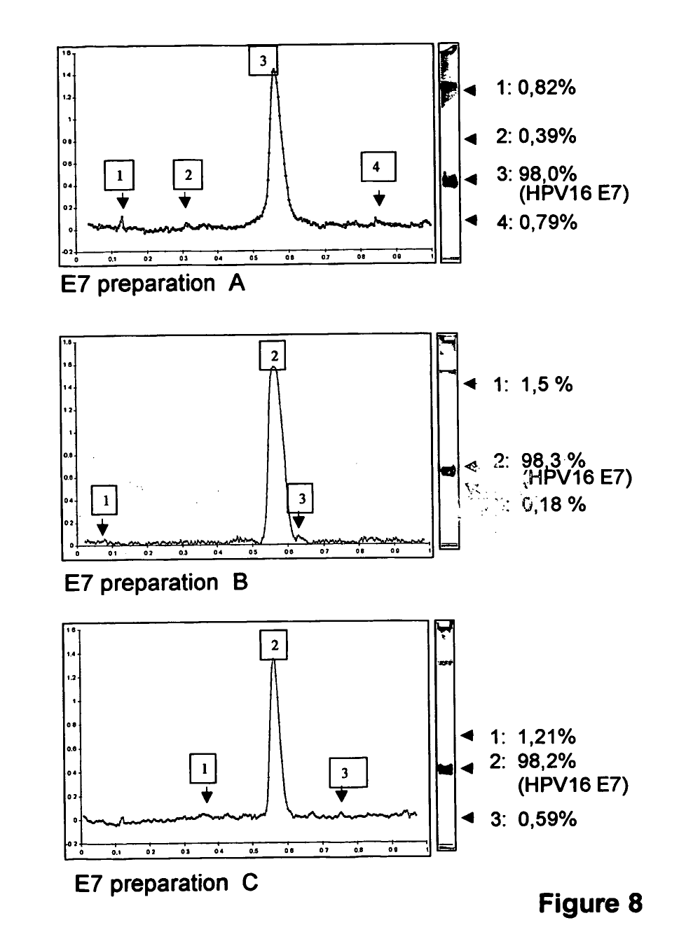

FIG. 8: Three different preparations (see appended example 1) of recombinantly expressed HPV 16 E7 protein were evaluated to detect, the purity of the preparations and the reproducibility of the applied methods. The amount of proteins separated per lane was 0,1 μg HPV16-E7 protein. The gel was silver stained according to Heukeshoven and Dernick (in R. Westermeier et al. 1990; ISBN 3-527-28172-X) for 30 min. The gel was scanned using a Fluor-S™ Multi-Imager system (BIORAD). Cross sections of defined lanes were saved as TIFF images using Quantity One (Quantitation Software by BIORAD). Evaluation of the gel-bands was performed by using TOTALLab evaluation software Version 1.1. The sum of all pixels over the entire length of one lane was assumed to be equivalent to 100% of protein applied (0.1 μg/lane). E7 concentration and purity was 98.0% (A), 98.3% (B) and 98.2% (C).

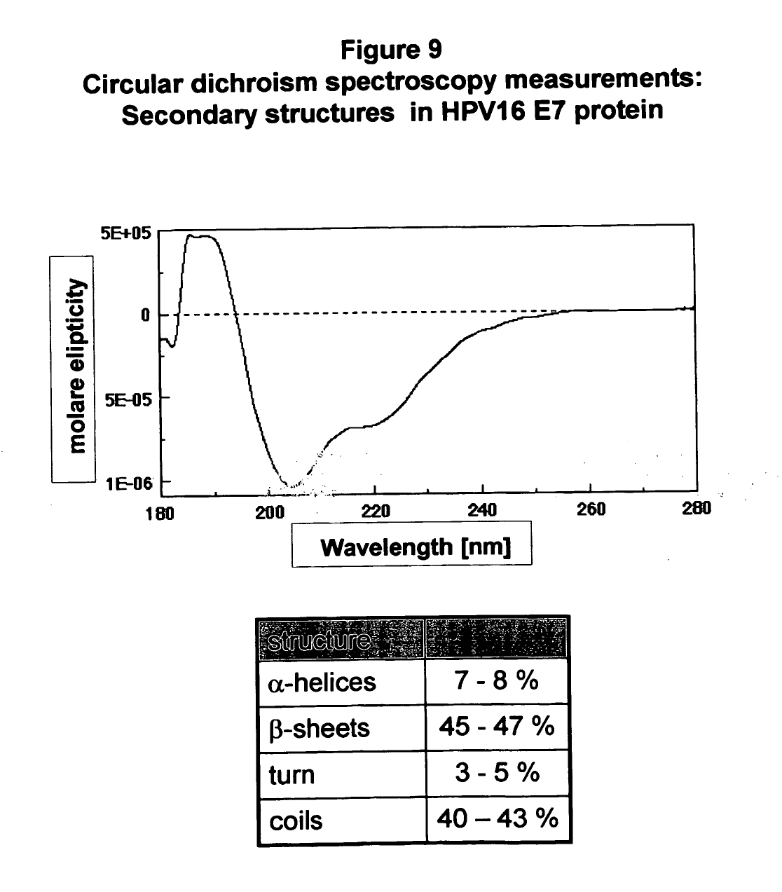

FIG. 9: A preparation of recombinantly expressed and highly purified HPV-16 E7 protein as described herein was evaluated for secondary structure elements in the native folded protein in a physiological solvent by CD spectroscopy. The native protein to be employed for immunization protocols is folded into secondary structure elements like β-sheets (45-47%), coils (40-43%), α-helices (7-8%) and turns (3-5%). Yet, also fragments of the native, highly purified E7 proteins as described herein may also be employed in immunization protocols.

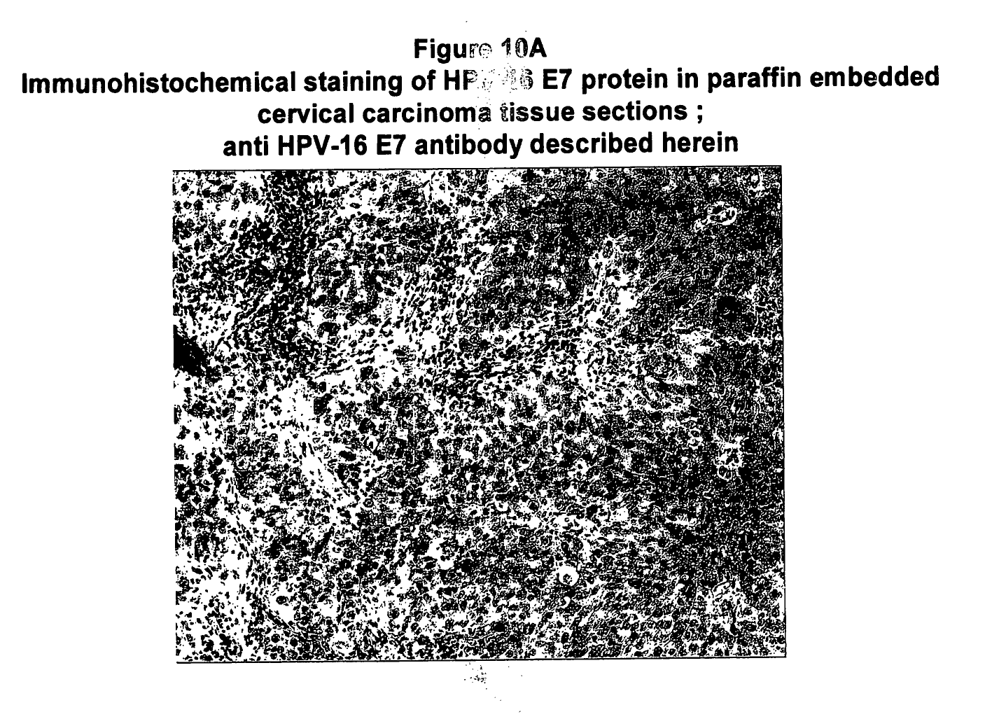

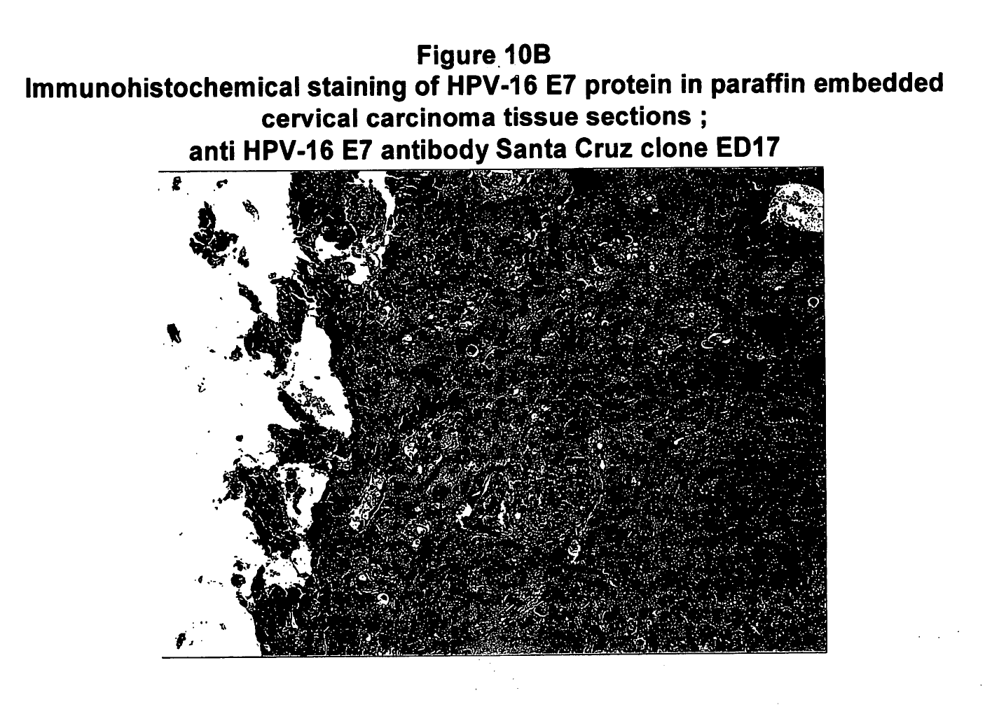

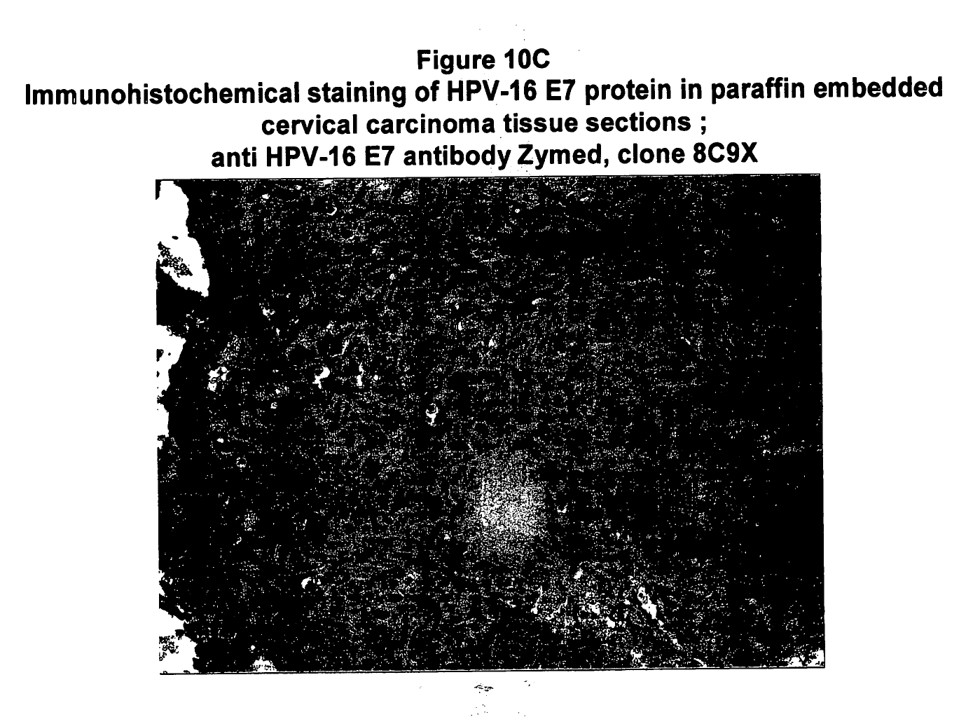

FIG. 10: Comparison of immunohistochemical staining of HPV-16 E7 protein in paraffin embedded cervical carcinoma tissue sections (consecutive sections from one tissue slice) by two different monoclonal anti HPV-16 E7 antibodies (Santa Cruz, Zymed) and the polyclonal anti HPV-16 E7 antibody described herein. Immunohistochemical staining was performed as described in Example 8. Antibodies was diluted according to manufacturers protocol. (A) In cervical carcinoma tissues anti HPV-16 E7 antibodies described herein stain virtually all cells in the tumor islets. (B,C) No clear signal was obtainable in cervical carcinoma tissues by the monoclonal anti HPV-16 E7 antibodies ED17 (Santa Cruz) and 8C9X (Zymed). In the latter cases, a high and apparently unspecific background is not restricted to the area that is cytologically recognized as tumor tissue, but were also present in the non-tumor tissue.

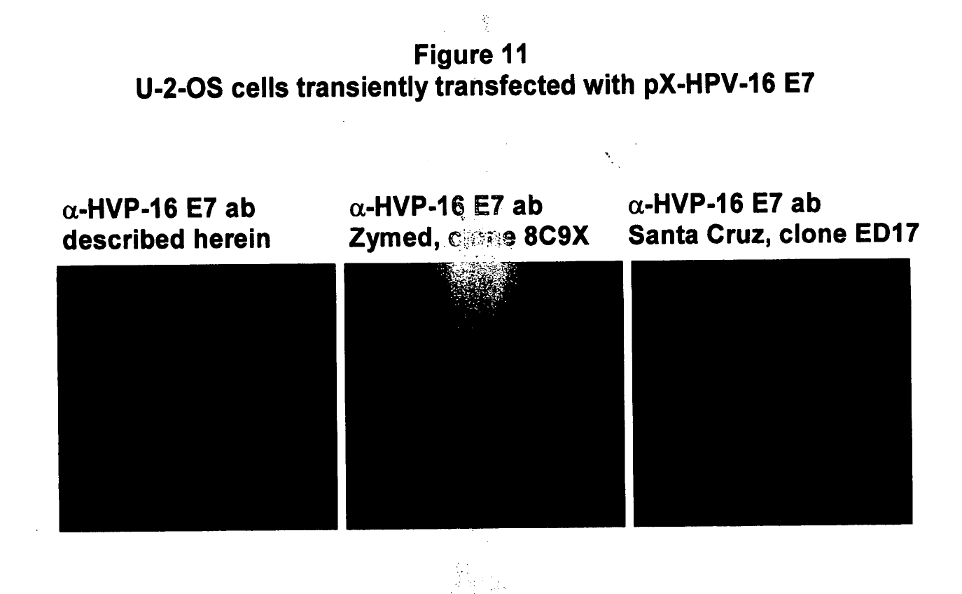

FIG. 11: Comparison of indirect immunofluorescence detection of HPV-16 E7 protein in transiently transfected U-2OS cells by the polyclonal anti HPV-16 E7 antibodies described herein and two commercialized monoclonal anti HPV-16 E7 antibodies (Santa Cruz, Zymed). The staining was performed as indicated in FIG. 3 and Example 7.

The invention is illustrated by the following examples:

### EXAMPLE 1

### Construction of the Bacterial Expression Vector for HPV-16 E7

The HPV-16 E7 oncogene was amplified from the vector pX-HPV-16 E7 (Mannhardt et al., 2000) by PCR using Pfu DNA polymerase as EcoRI repair/BamHI fragment. The sequence was inserted into the bacterial expression vector pET3a (Studier and Moffaft, 1986) prepared as NdeI repair/BamHI fragment generating the bacterial HPV-16 E7 expression vector pET3a-HPV-16 E7/clone 17. The sequence encoding for HPV-16 E7 was verified by sequencing.

### EXAMPLE 2

### Expression and Purification of Recombinant HPV-16 E7 Protein

The expression vector pET3a-HPV-16 E7/clone 17 was transformed into E. coli strain BL21 (DE3) pLysS. The bacteria were grown to OD₆₀₀=0.5 and the expression of the E7 protein was induced for 3 hours at 37C.° by adding IPTG (Biomol, Hamburg, Germany) to a final concentration of 0.4 mM. Bacteria were harvested by centrifugation for 10 minutes at 5 000×g. The cell pellets were frozen in 20 ml ice-cold lysis buffer (50 mM KCl, 20 mM H₂KPO₄ [pH 7.8], 50 mM DTT, 5% glycerol, 1μg/ml leupeptin, 1 mM PMSF, 1 mM NaF and 10 μg/ml Aprotinin) per liter bacterial culture. Cells were thawed on ice and lysed by sonication with glass beads (Sigma, Vienna, Austria) using a Sonifier 250 (Branson, Geneva, Switzerland). After centrifugation at 70 000×g for 1 hour, the supernatant was ammonium sulfate precipitated using 30% saturated (NH₄)₂SO4 solution. The (NH₄)₂SO4 pellet was dissolved in 4 ml MonoQ loading buffer (150 mM Tris/Cl pH 7.8, 10 mM NaCl, 10 mM DTT and 5% glycerol) and dialysed against MonoQ loading buffer. The dialysed probe was centrifuged at 10 000×g for 10 minutes and loaded onto a MonoQ HR10/10 column (Amersham Biosciences, Vienna, Austria) via a 10 ml SuperLoop (Amersham Biosciences, Vienna, Austria). Using a linear salt gradient the E7 protein eluted from the anion-exchange-column at 600 mM NaCl. 15 fractions were collected. The E7 containing fractions were pooled and loaded onto a HiLoad 16/60 Superdex 75 gel filtration column (Amersham Biosciences, Vienna, Austria) and run with the gelfiltration buffer (150 mM Tris/Cl pH 7.8, 150 mM NaCl and 10 mM DTT). At a flow of 0.5 ml/min. Gels were stained with Coomassie brilliant blue and the stained gel was evaluated by scanning, using the Adobe Photoshop Software and a Micro Tec Scan Maker 8700 Image Scanning Device. This generated a profile of relative optical density which was used to determined the integral corresponding to the E7 peak. With this software it is possible to calculate the percentage of total OD units which are represented by the E7 peak. As judged by densitometrical analysis the E7 fractions resulting from this run were more than 98% pure (e.g. analyzed by silver-stained SDS-PAGE), or more than >99.5% pure (e.g. judged by Coomassie-stained SDS-PAGE) and were concentrated using a Centriprep10 ultrafiltration filter (Amicon, Vienna, Austria). The identity of the E7-protein was confirmed by Western Blot (FIG. 1B) and through a peptide mass fingerprint (PMF) (FIG. 1C).

A further protocol for the purification of E7-protein comprises the following steps:

The expression vector pET3a-HPV-16 E7/clone 17 was transformed into E. coli strain BL21 (DE3) pLysS and preserved as glycerol stock. LB- or NZCYM-medium (25 ml) containing 100 μg/ml Ampicilline (Biomol, Hamburg, Germany;) and 25 μg/ml Chloramphenicol (Sigma, Vienna; Austria;) was inoculated with the glycerol stock and grown over night at 37° C. to a final OD₆₀₀ of 1.5. The next day NZCYB medium, containing 100 μg/ml Ampicillin and 25 μg/ml Chloramphenicol and 2 ml Glucose/I, was inoculated with 1% of the over night culture and grown at 37° C. to an OD₆₀₀ of 0.4. Culture volume was 400 ml per 2000 ml aeration flask. At OD₆₀₀=0.4 E7 expression was induced by adding IPTG (Biomol, Hamburg, Germany) to a final concentration of 0.4 mM. Two hours after induction bacteria were harvested by centrifugation for 10 minutes at 5 000×g. The drained cell pellets were either stored at −80° C. until further use (up to 3 month) or redissolved in ice-cold lysis buffer (50 mM KCl, 20 mM H₂KPO₄ [pH 7.8], 50 mM DTT, 5% glycerol, 1-μg/ml leupeptin, 1 mM PMSF, 1 mM NaF and 10 μg/ml Aprotinin) at a ratio of 2 ml fresh lysis buffer per pellet derived from 100 ml bacterial culture. When the pellet had been stored at −80° C. lysis buffer was added directly to the frozen material and cells were thawed on ice. For the following purification procedure two pellets from 400 ml E. coli culture each were used. Pellets were redissolved by repeated pipetting and lysed by sonication with glass beads (Sigma, Vienna, Austria) using a Sonifier 250 (Branson, Geneva, Switzerland) on ice. The sonified lysate was centrifuged at 70 000×g for 1 hour and the supernatant stored on ice. The remaining pellet was redissolved in lysis buffer (again 2 ml fresh lysis buffer per pellet derived from 100 ml bacterial culture) and sonified and centrifugated as stated above. Supernatants were pooled, cooled on ice and subjected to a two-step ammonium sulphate precipitation procedure.

To prepare a saturated Ammonium sulphate solution 75 g of (NH₄)₂SO₄ were added to 100 ml of 50 mM. NaCl, 150 mM Tris/HCl pH 7.8. Ammonium sulphate was dissolved at RT and the saturated solution was cooled down on ice. (The cooled solution contained a few crystals of precipitated (NH₄)₂SO₄ indicating 100% saturation.) The saturated (NH₄)₂SO₄ solution was prepared freshly prior to use.

The lysate was made 15% ammonium sulphate by adding 15 parts of cold, saturated (NH₄)₂SO₄ solution to 85 parts of cold lysate (5.65 ml saturated (NH₄)₂SO₄ to 32 ml lysate). The mixture was stirred gently on ice for 30 min and centrifuged at 4° C. for 30 min at 30 000×g. The supernatant (i.e. 15% (NH₄)₂SO₄) was removed and made 38% ammonium sulphate by adding 1 part of cold, saturated (NH₄)₂SO₄ solution to 2.7 parts of cold supernatant (14 ml saturated (NH₄)₂SO₄ to 37.65 ml supernatant). The mixture was stirred gently on ice for 30 min and centrifuged at 4° C. for 30 min at 30 000×g.

After discharging the supernatant, the pellet was dissolved in dialysis buffer (150 mM Tris/HCl pH 7.8, 10 mM NaCl, 10 mM DTT and 5% glycerol) at a ratio of 1 ml per pellet derived from 100 ml bacterial culture. Dialysis was performed at 4° C. for 12 hours applying 3 buffer changes. Dialysis tubings with a molecular weight cut-off of 10 000 dalton were used; the total volume of dialysis buffer was 250 times the sample volume. DTT was added prior to every buffer change. (For 8 ml of dissolved (NH₄)₂SO₄-pellet, 3×670 ml dialysis buffer were used). The dialysed probe was centrifuged at 10 000×g for 10 min and the supernatant loaded (flow=1 ml/min) onto a MonoQ HR 10/10 anion-exchange column (Amersham Biosciences, Vienna, Austria) equilibrated to 10% MonoQ buffer B (MonoQ buffer A: 150 mM Tris/HCl pH 7.8, 10 mM DTT (added prior to use) and 5% glycerol; MonoQ buffer B: 150 mM Tris/HCl pH 7.8, 1M NaCl, 10 mM DTT added prior to use) and 5% glycerol;). The MonoQ column was washed with 2 column volumes (CV) (flow=4 ml /min) 10% buffer B, and eluted in multi step gradient at a flow rate of 2 ml/min: 10% -47% B (2 CV), 47% B (2CV), 47% -100% B (2 CV). At 47% buffer B (470 mM NaCl) E7 eluted in a prominent peak over 3 fractions of 1 ml each. E7 containing fractions were individually loaded onto a HiLoad 16/60 Superdex 75 gel filtration column (Amersham Bioscienees, Vienna, Austria) and eluted at a flow rate of 0.5 ml/min with the gelfiltration buffer (150 mM Tris/HCl pH 7.8,150 mM NaCl and 10 mM DTT (added prior to use)); fraction volume was 2 ml. E7 containing fractions from 3 runs were controlled on SDS-PAGE followed by coomassie stain. E7 fractions of highest purity were pooled and the protein concentration was determined according to Bradford. The pool was diluted with gelfiltration buffer to a final concentration of 1 mg/ml and frozen in aliquots for further use. The total yield from 800 ml E. coli culture was approximately 14 mg of native, highly purified HPV-E7 in NMR-grade.

A further protocol for the purification of E7-protein comprises the following steps:

The expression vector pET3a-HPV-16 F-E/clone 17 was transformed into E. coli strain BL21 (DE3) pLysS and preserved as glycerol stock. LB- or NZCYM-medium (25 ml) containing 100 μg/ml Ampicilline (Biomol, Hamburg, Germany;) and 25 μg/ml Chloramphenicol (Sigma, Vienna; Austria;) was inoculated with the glycerol stock and grown over night at 37° C. to a final OD₆₀₀ of 1.5. The next day, 99 parts of NZCYB medium, containing 100 μg/ml Ampicillin and 25 μg/ml Chloramphenicol and 2 ml Glucose/I, was inoculated with 1 part of the over night culture and grown at 37° C. to an OD₆₀₀ of 0.4. Culture volume was 400 ml per 2000 ml aeration flask. At OD₆₀₀=0.4 E7 expression was induced by adding IPTG (Biomol, Hamburg, Germany) to a final concentration of 0.4 mM. Two hours after induction bacteria were harvested by centrifugation for 10 minutes at 5 000×g. The drained cell pellets were either stored at −80° C. until further use (up to 3 month) or redissolved in ice-cold lysis buffer (50 mM KCl, 20 mM H₂KPO₄ [pH 7.8], 50 mM DTT, 5% glycerol, 1-μg/ml leupeptin, 1 mM PMSF, 1 mM NaF and 10 μg/ml Aprotinin) at a ratio of 2 ml fresh lysis buffer per pellet derived from 100 ml bacterial culture. When the pellet had been stored at −80° C. lysis buffer was added directly to the frozen material and cells were thawed on ice. For the following purification procedure two pellets from 400 ml E. coli culture each were used. Pellets were redissolved by repeated pipetting and lysed by sonication with glass beads (Sigma, Vienna, Austria) using a Sonifier 250 (Branson, Geneva, Switzerland) on ice. The sonified lysate was centrifuged at 70 000×g for 1 hour and the supernatant stored on ice. The remaining pellet was redissolved in lysis buffer (again 2 ml fresh lysis buffer per pellet derived from 100 ml bacterial culture) and sonified and centrifugated as stated above. Supernatants were pooled, cooled on ice and subjected to an ammonium sulphate precipitation procedure.

The lysate was made 38% ammonium sulphate by adding 38 parts of cold, saturated (NH₄)₂SO₄ solution to 62 parts of cold lysate (19.6 ml saturated (NH₄)₂SO₄ to 32 ml lysate). The mixture was stirred gently on ice for 30 min and centrifuged at 4° C. for 30 min at 30 000×g. After carefully discharging the supernatant, the pellet was dissolved in dialysis buffer (150 mM Tris/HCl pH 7.8, 10 mM NaCl, 10 mM DTT and 5% glycerol) at a ratio of 1 ml per pellet derived from 100 ml bacterial culture.

Dialysis was performed at 4° C. for 12 hours applying 3 buffer changes. Dialysis tubings with a molecular weight cut-off of 10 000 dalton were used; the total volume of dialysis buffer was 250 times the sample volume. DTT was added prior to every buffer change. (For 8 ml of dissolved (NH₄)₂SO₄-pellet, 3×670 ml dialysis buffer were used). The dialysed probe was centrifuged at 10 000×g for 10 min and the supernatant loaded (flow=1 ml/min) onto a MonoQ HR 10/10 anion-exchange column (Amersham Biosciences, Vienna, Austria) equilibrated to 10% MonoQ buffer B (MonoQ buffer A: 150 mM Tris/HCl pH 7.8, 10 mM DTT (added prior to use) and 5% glycerol; MonoQ buffer B: 150 mM Tris/HCl pH 7.8, 1M NaCl, 10 mM DTT added prior to use) and 5% glycerol;). The MonoQ column was washed with 2 column volumes (CV) (flow=4 ml/min) 10% buffer B, and eluted in multi step gradient at a flow rate of 2 ml/min: 10% -47% B (4 CV), 47%B (2CV), 47%-100% B (2 CV). At 47% buffer B (470 mM NaCl) E7 eluted in a prominent double peak over 4 fractions of 1 ml each. E7 containing fractions were individually loaded onto a HiLoad 16/60 Superdex 75 gel filtration column (Amersham Bioscienees, Vienna, Austria) and eluted at a flow rate of 0.5 ml/min with the gelfiltration buffer (150 mM Tris/HCl pH 7.8, 150 mM NaCl and 10 mM DTT (added prior to use)); fraction volume was 2 ml. E7 containing fractions from 4 runs were controlled on SDS-PAGE followed by coomassie stain. E7 fractions of highest purity were pooled and the protein concentration was determined according to Bradford. The pool was diluted with gelfiltration buffer to, a final concentration of 1 mg/ml and frozen in aliquots for further use. The total yield from 800 ml E. coli culture was approximately 14 mg of native, highly purified HPV-E7 in NMR-grade.

Three different preparations of recombinantly expressed HPV 16 E7 protein were evaluated to document, the purity of the preparations and the reproducibility of the applied methods. Material generated on Aug. 8^{th,} 2002 (used to immunise chinchilla rabbits; preparation “A”), and 2 production lots from Dec. 17^{th,} 2002 (lot 1 used to immunise goats; lot 2 used to prepare an E7-affinity column; preparations “B” and “C”) were run on a 12.5% SDS-PAGE under reducing conditions (2.5% β-Mercaptoethanol). The amount of proteins separated per lane was 0.1 μg HPV16-E7, determined according to Bradford, using BSA as standard. The gel was silver stained according to Heukeshoven and Dernick (in R. Westermeier et al. 1990; ISBN 3-527-28172-X) for 30 min. The gel was scanned using a Fluor-S™ Multi-Imager system (BIORAD). Cross sections of defined lanes were saved as TIFF images using Quantity One (Quantitation Software by BIORAD). Evaluation of the gel-bands was performed by using TOTALLab evaluation software Version 1.1.

FIG. 8 shows the results from densitometric evaluation of three independent preparations A, B and C. Results were calculated from separation gels. A light background-staining in the stacking gel, derived from the sample buffer, was observed. Since the light background staining on top of the separation gel was found in every lane, it is assumed to be derived from an irrelevant compound from the sample buffer. Prior to evaluation, the background was subtracted from each lane separately. The sum of all pixels over the entire length of one lane was assumed to be equivalent to 100% of protein applied (0.1 μg/lane). Peaks were evaluated be recalculating the pixel-intensity of every protein band found into % of the total protein amount per lane. E7 concentration was 98.0% (A), 98.3% (B) and 98.2% (C). The curves shown in FIG. 8 are original traces from scanned lanes exported as MS-Excel files as the used set-up did not allow to print evaluated curves directly.

Circular Dichroism spectroscopy (CD) Measurements of HPV-16 E7 Protein in Solution

Circular Dichroism (CD) is observed when optically active matter absorbs left and right hand circular polarized light slightly differently. CD spectra for distinct types of secondary structure present in peptides, proteins and nucleic acids are different. The analysis of CD spectra can therefore yield valuable information about secondary structure of biological macromolecules. In our case Circular Dichroism Spectroscopy is used to gain information about the secondary structure of native proteins and polypeptides in solution. The CD is a function of wavelength and is measured with the CD spectropolarimeter JASCO J-715. (See Circular Dichroism and Optical Rotary Dispersion of Proteins and Polypeptides, A. J. Alder, N. J. Greenfield and G. D. Fasman, Meth. Enzymology 27, 675 (1973)).

A preparation of recombinantly expressed and highly purified HPV-16 E7 protein as described herein was further evaluated for secondary structure elements that will occur in the native folded protein in a physiological solvent. Because of 7 Cysteins in the E7 molecule, the E7 protein tends to build di- and multimeres with proteins in vicinity by disulfide bridges. Patrick et al, 1992, JBC 265 (10):6910, describes, that no such disulfide-bonds exist inside the native E7 molecule. It was demonstrated that 3 cysteins are accessible to solvent, while cysteins in the two concerved Cys-X-X-Cys motifs are likely involved to be part of a zinc-finger motif. For this reason inclusion of a reducing substance in solvent like DTT (or 2-ME) results in monomeric native E7 protein particles (DTT and 2-ME do not have any denaturing effect). In addition, the amount of DTT in CD measurement is diluted to the lowest concentration that might be possible to adhere reducing conditions. Repeated measurements was carried out in 8 μl of a 50-100 μmolar protein solution in NMR buffer (20 mM H₂KPO₄, 50 mM KCl, 10 mM NaCl, 10 mM DTT, pH 7.5) diluted in in 80 μl of a. dest.

The obtained measurement data were interpreted by the calculation program of the CD spectropolarimeter JASCO H-715 (H-700 Secondary Structure Estimation for Windows, version 1.10.02, Jasco). The CD-spectrum and structural data are shown in FIG. 9. The HPV-16 E7 protein is folded into secondary structure elements like β-sheets (45-47%), coils (40-43%), α-helices (7-8%) and turns (3-5%).

### EXAMPLE 3

### Generation of Polyclonal HPV-16 E7 Antibodies:

Purified preparations of the HPV-16 E7 protein were used to produce highly specific polyclonal anti-HPV-16 E7 antibodies in chinchilla bastard rabbits (Charles River, Germany). 1^{st} injection: 700 μl complete Freund's adjuvant (Sigma, Vienna, Austria) was mixed with 500 μg HPV-16 E7 protein dissolved in 700 μl PBS by sonication (Branson sonifier 250, level 5-7, 3×10 seconds). A total of 300 μg HPV-16 E7 protein was injected. 1^{st} boost: 32 days after the first injection, 500 μl incomplete Freund's adjuvant was mixed with 500 μg 16 E7 protein dissolved in 500 μl PBS by sonication. A total of 500 μg HPV-16 E7 protein was injected. 2^{nd} boost: 28 days after the first boost, 500 μl incomplete Freund's adjuvant was mixed with 500 μg 16E7 protein dissolved in 500 μl PBS by sonication and a total of 500 μg HPV-16 E7 protein was injected. 3^{rd} boost: 27 days after the second boost, 500 μl incomplete Freund's adjuvant was mixed with 500 μg 16 E7 protein dissolved in 500 μl PBS by sonication. A total of 500 μg HPV-16 E7 protein was injected. Bleeding was done 10 days after the third boost. In particular, small aliquots of sera were tested in western blot 10 days after the first, second and third boost (second, third and forth injection). A first and clear signal was obtained after the third boost.

| day | application of HPV16 E7 | bleedings |
|---|---|---|
| −3 | | pre-immune |
| | | serum taken |
| 1 | immunisation with 500 μg 16 E7 incomplete | |
| | FA | |
| 33 | 1^{st} boost with 500 μg 16 E7 in incomplete FA | |
| 43 | | 1^{st} test bleeding |
| 61 | 2^{nd} boost with 500 μg 16 E7 in incomplete FA | |
| 71 | | 2^{nd} test bleeding |
| 88 | 3^{rd} boost with 500 μg 16 E7 in incomplete FA | |
| 98 | | final bleeding |

A good immune response was also achieved by using 150 μg and 300 μg HPV16 E7 as antigen respectively. The immunisation schedule was as stated above.

A further protocol for the generation of a polyclonal HPV-16 E7 antibody comprises the generation of said antibody/serum in goat. Said generation was carried out as follows:

Highly purified preparations of the HPV-16 E7 protein (see example 2) were used to produce highly specific polyclonal anti-HPV-16 E7 antibodies in goats. 1^{st} injection: 1100 μl complete Freund's adjuvant (Sigma, Vienna, Austria) was mixed with 1000 μg HPV-16 E7 protein dissolved in 1000 μl G75 gel filtration buffer (example 2:150 mM Tris/HCl pH 7.8, 150 mM NaCl and 10 mM DTT) according to a “syringe-method” (Oxf. Univ. Press; 2000: Practical approach series; ISBN 0-19-963711-3 Vol. Immunoassays; Edited by J. P. Gosling; p.28). A total of 1000 μg HPV-16 E7 protein was injected. 1^{st} boost: 28 days after the first injection, 1100 μl complete Freund's adjuvant was mixed with 1000 μg 16 E7 protein dissolved in 1000 μl G75 gel filtration buffer by the syringe-method. A total of 1000 μg HPV-16 E7 protein was injected. 2^{nd} boost: 28 days after the first boost, 1000 μl incomplete Freund's adjuvant was mixed with 1000 μg 16E7 protein dissolved in 1000 μl G75 gel filtration buffer by the syringe-method. A total of 1000 μg HPV-16 E7 protein was injected. 3^{rd} boost: 28 days after the second boost, 1000 μl G75 gel filtration buffer by the syringe-method. A total of 1000 μg HPV-16 E7 protein was injected. Small aliquots of sera were tested in western blot 10 days after the first, second and third boost (second, third and forth injection). Final bleeding was done 14 days after the third boost. A first and clear signal was obtained after the third boost.

| day | application of HPV16 E7 | bleedings |
|---|---|---|
| −3 | | pre-immune |
| | | serum taken |
| 1 | immunisation with 1000 μg 16 E7 incomplete | |
| | FA | |
| 29 | 1^{st} boost with 1000 μg 16 E7 incomplete FA | |
| 39 | | 1^{st} test bleeding |
| 57 | 2^{nd} boost with 1000 μg 16 E7 in incomplete FA | |
| 67 | | 2^{nd} test bleeding |
| 85 | 3^{rd} boost with 1000 μg 16 E7 in incomplete FA | |
| 95 | | 3^{rd} test bleeding |
| 99 | | final bleeding |

### EXAMPLE 4

### AffinityPurification of Polyclonal HPV-16 E7 Antibodies

Glutathione-S-transferase (GST-HPV-16 E7) and GST (control) were expressed from the expression vectors pGEX4T-GST-HPV-16 E7 and pGEX4T (Mannhardt, 2000; Mol Cell Biol 20:6483-95) in the E. coli strain DH5α. Expression was induced by adding IPTG to a final concentration of 1 mM to a 200 ml bacterial culture at OD₆₀₀=1.0. The bacteria were washed once in PBS and lysed in PBSDT (1.5 mM KH₂PO₄, 8.1 mM Na₂HPO₄ [pH 7.4], 2.7 mM KCl, 137 mM NaCl, 0.2 mM phenylmethylsulfonyl fluoride [PMSF], 1 mM NaF, 1 mM dithiothreitol [DTT] and 0.5% Triton X-100) by sonication using a Branson sonifier 250. The lysates were centrifuged at 4 000×g for 10 minutes and afterwards at 30 000×g for 30 minutes to remove the cell debris. The recombinant proteins were purified by affinity-chromatography using the glutathione sepharose 4B system (Amersham, Vienna, Austria). Clear supernatants were incubated for 3 hours at 4° C. with 150 μl glutathione sepharose 4B beads, which were prior, equilibrated in cold (4° C.) PBSDT. After the binding interval the beads were washed 4 times in 5 ml of PBSDT and stored at 4° C. in PBSDT. Purity of the preparation was controlled by western blotting using an anti E7 antibody (clone ED17, Santa Cruz, Vienna, Austria) and by Coomassie staining. Aliquots of 200 μg of bound GST proteins were separated on a 12.5% SDS-PAGE and transferred to a polyvinylidene difluoride (PVDF) membrane (NEN, Boston, USA) by electro blotting. PVDF membranes were dried and the proteins were crosslinked to the membrane by UV irradiation. The protein bands were stained with Ponceau S solution, excised from the PVDF membrane, destained and transferred to microfuge tubes.

Subsequently, the fragments were incubated with the polyclonal rabbit HPV-16 E7 antiserum for 2 hours at room temperature and washed 3 times in PBS-T (1.5 mM KH₂PO₄, 8.1 mM Na₂HPO₄ [pH 7.4], 2.7 mM KCl, 137 mM NaCl and 0.5% (vol/vol) Tween 20). Each fragment was then eluted by three 30 seconds washes with 5 mM glycine-HCl, [pH 2.3], 500 mM NaCl, 0.5% (vol/vol) Tween 20, 10 μg/ml BSA in volumes of 500 μl; these eluates were immediately neutralized by the addition of Na₂PO₄ to a final concentration of 50 mM. The purified antibodies were concentrated 4 times using a centriprep YM-3 centrifugal filter (Millipore Corporation, Bedford, USA). After the pH 2.3 elution, fragments were further washed with three similar aliquots of PBS-T and 10 μg/ml of BSA, followed by three washes with 3 M NH₄SCN, 150 mM KCl, 10 mM NaPO₄ [pH 6.0], and 10 mg/ml BSA. The procedure was repeated 5 times

A further protocol for affinity purification of polyclonal HPV-16 E7 antibodies comprises the following:

Three different columns were used to purify polyclonal HPV-16 E7 antibodies from animal serum by affinity chromatography.

Column 1: (column to purify total IgG from antiserum). A HiTrap Protein G HP column (Amersham Biosciences, Vienna, Austria) was used according to the manufacturers protocol to isolate total IgG from antiserum.

Column 2: (pre-column without antigen to adsorb unspecific antibodies to the affinity matrix): 2.8 g of freeze dried CNBr-activated sepharose 4B (Amersham Bioscieces, Vienna, Austria) were activated according to the manufacturers protocol and transferred into coupling buffer (100 mM NaHCO₃, 500 mM NaCl, pH 8.3) containing 13 mg of NIH 3T3 fibroblasts cell lysate (determined according to Bradford). Coupling was performed for 2 hours at room temperature in a 50 ml Falcon tube attached to a rotating platform. Once coupling was completed, the affinity matrix was packed into a XK16 FPLC column (Amersham Biosciences, Vienna, Austria) by gravity. The settled gel-bed (10 ml) was then washed with 5 column volumes of coupling buffer and 5 column volumes of blocking buffer (1M ethanolamine, pH 8.0). The column was then left at room temperature for 2 hours with out agitation to block remaining active groups, and thereafter washed with 5 column volumes high pH buffer (100 mM Tris/HCl, 500 mM NaCl, pH 8.0) and 5 column volumes low pH buffer (100 mM Na-acetate, 500 mM NaCl, pH 4.0). The cycle high pH-wash/low pH-wash was repeated 5 times. Finally the column was attached to the FPLC system and equilibrated to running buffer (PBS, 200 mM NaCl, 5 mM EDTA, 0.05% NaN3, pH 7.4). Protein contents (Bradford) of coupling buffer before and after coupling, and of all through-runs and wash buffers collected, revealed a coupling efficiency of approximtatly 80% of NIH-3T3 proteins to the column. The ligand density was 1 mg/ml gel-bed; the column volume was 10 ml.

Column 3: (affinity column, carrying purified HPV16-E7 protein to isolate polyclonal HPV16-E7 antibodies). As affinity matrix CNBr-activated Sepharose 4B (Amersham Biosciences, Vienna, Austria) was used. Preparation of the column was as stated above (column 2), but with recombinant, purified HPV16-E7 (examples 2A-2C) used as ligand. Prior to coupling, the ligand was dialysed (from 150 mM Tris/HCl, 150 mM NaCl, 10 mM DTT, pH 7.8) into coupling buffer as Tris would interfere with the coupling procedure. The optimal ligand density for affinity purification was found to be 1 mg antigen per ml gel-bed. For the experiment described below, an affinity column of a bed-volume of 3 ml, carrying 1.5 mg of HPV16-E7 was used.

Purification of the Polyclonal HPV16-E7 Antibody:

Antiserum was diluted 1+9 in running buffer (PBS, 200 mM NaCl, 5 mM EDTA, 0.05% NaN₃, pH 7.4). Diluted material was filtered trough a 0.45 μm sterile filter and passed over a 1 ml Protein G column using an Akta Prime system (Amersham Biosciences) at a flow rate of 1 ml/min. The column was extensively washed with running buffer until the baseline was zero (5 ml/min). Total IgG was eluted in 1 ml fractions (1 ml/min) with 100 mM Glycine, 0.05% NaN₃, pH 2.5 into 1.5 ml reaction vials containing 50 μl of 3 M KH₂PO₄/K₂HPO₄-buffer pH 7.4 to neutralize the low pH of the elution buffer.

IgG containing fraction (4×1.050 ml) were pooled, topped up to 10 ml with running buffer and loaded onto column 2 equilibrated with 10 column volumes of running buffer. Material was passed over the pre-column at a flow rate of 5 ml/min for 60 min in a closed circle to remove antibodies that would bind unspecifically to the CNBr activated sepharose matrix, and to immobilised proteins other than HPV16-E7. Thereafter the adsorbed material was collected (still 10 ml) and pooled with 5 column volumes of running buffer used to wash loosely bound, but probably specific antibodies from the pre-column. Finally the material (15 ml) was passed over the affinity column (column 3, equilibrated in running buffer) at a flow rate of 5 ml/min in a closed circle until an equilibrium was reached. The column was then washed (5 ml/min) with PBS, 1M NaCl, 5 mM EDTA, 0.05% NaN₃, pH 7.4 until the baseline reached zero. After re-equilibration into running buffer (10 column volumes), polyclonal anti HPV16-E7 antibodies were eluted (1 ml/min) with 100 mM Glycine, 0.05% NaN₃, pH 2.5 into 1.5 ml reaction vials containing 50 μl of 3 M KH₂PO4/K₂HPO₄ buffer to neutralize the low pH of the elution buffer (fraction size was 1 ml). After elution, the column pH was set back to neutral by passing 10 column volumes of 1M Tris/HCl pH 7.4, 10 column volumes of 3 M KSCN, 150 mM KCl, 10 mM KH₂PO₄/K₂HPO₄, pH 7.4 and 20 column volumes of running buffer through the system.

Anti HPV16-E7 antibody containing fractions were pooled and tested further like stated below. It was found that the pre-column (column 2, carrying NIH-3T3 cell lysate as ligand) in some cases could be omitted. Pooled eluates from column 1 (protein G column) were diluted 1+9 in running buffer and directly applied to column 3 (affinity column).

### EXAMPLE 5

### Test of the Affinity Purified HPV-16 E7 Antibodies

The affinity purified HPV-16 E7 antibodies specifically recognize HPV-16 E7 in cell lysates from HPV-16 E7 expressing mammalian cells in western blot experiments (FIG. 1). HPV-16 E7 was also detectable in human U-2-OS cells transiently transfected with a HPV-16 E7 expression vector by indirect immunofluorescence microscopy using the confocal scanning system (FIG. 2). Furthermore, the antibodies recognize HPV-16 E7 in immunohistochemical experiments done in paraffin-embedded sections of cervical carcinomas derived from biopsies of HPV-16 positive patients (FIG. 3). Biopsies from 12 carcinoma patients were analyzed and positive signals were obtained with the E7 antibody in all 12 cases. Furthermore, in two cases of cervix biopsies which had previously been classified by PCR-methods as “HPV-16 negative”, the antibody described herein was able to specifically detect expressed E7.

### EXAMPLE 6

### Western Blot (Immunoblot) Analysis

Cell extracts were separated on a 12.5% sodium dodecyl sulfate (SDS)-polyacrylamide gel, and proteins were transferred to a PVDF membrane (NEN, Boston, USA). The membrane was incubated in blocking buffer (0.05% Tween 20/5% low fat milk powder in PBS) for 1 hour at room temperature, washed in blocking buffer and incubated with the first antibody (affinity purified polyclonal rabbit anti-HPV-16 E7 antibody) for 1 hour at room temperature. After washing in blocking buffer, PBS/0.05% Tween 20 and PBS/5% low fat milk powder the membrane was incubated with the second antibody (peroxidase-conjugated anti-rabbit IgG, Promega, Mannheim, Germany) for 45 minutes at room temperature. The membrane was washed, and the bound antibodies were visualized by using the chemiluminescence Western blotting detection system (NEN, Boston, USA).

### EXAMPLE 7

### Indirect Immunofluorescence Analysis

U-2-OS cells were cultured in DMEM+10% FCS. For transient expression of cDNAs, cells were grown to about 80% confluence on glass coverslips coated with 0.05% gelatin. Transfection of the expression vector pJ4HPV-16 E7 (Massimi, et al., (1997) J. Gen. Virol. 78, 2607-2613) was performed by using Effectene (Qiagen, Hilden, Germany). 24 h post-transfection, cells were prepared for indirect immunofluorescence according to standard protocols, including methanol fixation. After incubation with the primary antibody (affinity purified polyclonal rabbit anti-HPV-16 E7 antibody), and secondary antibody (FITC-conjugated anti-rabbit IgG, Dianova, Hamburg, Germany), cells were washed and embedded in Fluoromount G (Biozol, Eching, Germany). Samples were viewed by indirect immunofluorescence microscopy using the confocal scanning system MicroRadiance (Bio-Rad, Munich, Germany) in combination with a Zeiss Axiophot microscope. The following filters were used for FITC-derived fluorescence: excitation at 488 nm, emission at 515-530 nm). In these experiments, HPV-16 E7 were detected in 5-10% of the transfected cells, whereas no signal was obtained in cells that have been transfected with the empty expression vector. This result clearly proves that the antibody is specific for the E7 protein and does not detect any non-specific background under these conditions. The superior properties of the antibodies of the invention can be furthermore illustrated in the following experiment: To calibrate the antibodies, human U2OS cells were transfected with a CMV-driven expression vector for HPV-16 E7 and the staining of transfected cells by the antibodies as described herein was compared to the staining pattern obtained with commercially available antibodies from SantaCruz Biotechnology (ED17) or from Zymed Laboratories (8C9X). Staining was analysed by indirect immunofluorescence and evaluated by confocal microscopy. In these experiments, the commercially available antibodies (obtained from SantaCruz Biotechnology and Zymed Laboratories) were employed in accordance with manufacturers recommendations and gave high, unspecific background staining in all cells. Yet, no specific signal for detection of E7 antigene in the transfected cells could be obtained with these prior art antibodies. The corresponding results are documented in appended FIGS. 10 and 11. In contrast, the antibodies according to the invention are able to specifically detect expressed E7 (positive signals in 5-10% of the transfected cells) and reveal no signal in cells transfected by an empty expression vector. These results indicate that the antibodies described herein recognize only the E7 protein, whereas the commercially available antibodies used in the study recognize unrelated antigens in the preparation. When tested in immunohistochemical stainings, there was a high and apparently non-specific background obtained with the antibodies obtained from SantaCruz Biotechnology or Zymed Laboratories in tissues derived from cervix carcinoma patients, as well as in tissue derived from normal cervix. Furthermore, the positive signals obtained with the SantaCruz antibodies were not restricted to the area that is cytologically recognized as tumor tissue, but were also present in the non-tumor tissue. In contrast to these results, staining of the biopsy material by the antibody according to the invention yielded positive results only for HPV-16 positive patients. As can be seen in FIG. 4, staining was clearly confined to the area of the tumor.

### EXAMPLE 8

### Immunohistochemical Detection of HPV-16 E7 in Biopsies Derived from Cervical Carcinomas

Immunohistochemistry was performed on paraffin-embedded sections of HPV16 positive biopsies derived from cervical carcinomas and control tissue specimens. The paraffin-embedded tissue specimens were sectioned at 5 μm. Sections were mounted on slides, deparaffinized in xylol (2×10 minutes), incubated for 5 minutes each in 100%, 90%, 80% and 70% ethanol and blocked in 5% H₂O₂ in absolute methanol for 15 minutes. Before immunostaining the sections were washed twice in TRIS buffer (7.75 g Tris-HCl pH 7.5, 8.78 g NaCl ad 1 liter aqua dest) and processed for a 15 minutes blocking reaction in diluted (1:10 in TRIS buffer/1% BSA) goat-serum (DAKO, Hamburg, Germany). Sections were washed in TRIS/1% BSA buffer and incubated with the first antibody (affinity purified polyclonal rabbit anti-HPV-16 E7 antibody) for 1 hour at room temperature in buffer B (10 μg/ml BSA/10 μg/ml NIH3T3 lysate in PBS). The samples were rinsed twice in TRIS/1% BSA buffer and incubated for 1 hour at room temperature with the second antibody (Biotin-conjugated anti-rabbit IgG, DAKO, Glostrup, Denmark). After the washing step in TRIS/1% BSA, ExtrAvidin-conjugated peroxidase solution (Amersham Biosciences, Vienna, Austria) was added and the samples were incubated for 1 hour at room temperature, rinsed in TRIS buffer and processed for staining. Bound antibodies were visualized with DAB (3.3′-diaminobenzidine) (Sigma, Vienna, Austria) as substrate chromogen. Slides were counterstained with Hemalaun and coverslipped using Eukitt (Merck; Darmstadt, Germany). Brightfield microscopy with photography was performed using a Leica DMRB microscope and a Nikon Coolpix 995 camera.

A further protocol comprises the following steps:

Immunohistochemistry was performed on paraffin-embedded sections of HPV-16 positive biopsies derived from cervical carcinomas and control tissue specimens. The paraffin-embedded tissue specimens were sectioned at 2 and 5 μm. Sections were mounted on slides, deparaffinized in xylol (2×10 minutes), incubated for 5 minutes each in 100%, 90%, 80% and 70% ethanol and blocked in 5% H₂O₂ in absolute methanol for 15 minutes. Before immunostaining the sections were washed twice in TRIS buffer (7.75 g Tris-HCl pH 7.5, 8.78 g NaCl ad liter aqua dest/0.1% Tween 20) and processed for a 15 minutes blocking reaction in diluted (1:10 in TRIS buffer/1% BSA, 0.1% Tween 20) goat serum (DAKO, Hamburg, Germany). Sections were washed in TRIS/1% BSA/0.1% Tween 20 buffer and incubated with the first antibody (affinity purified polyclonal rabbit anti-HPV-16 E7 antibody) for 1 hour at room temperature in buffer B (10 μg/ml BSA/10 μg/ml NIH3T3 Lysate , 0.1% Tween 20 in PBS). The samples were rinsed twice in TRIS/1% BSA/0.1% Tween 20 buffer and incubated for 1 hour at room temperature with the second antibody (Biotin-conjugated anti rabbit IgG, DAKO, Glostrup, Denmark). After the washing step in TRIS/1% BSA, ExtrAvidin-conjugated peroxidase solution (Amersham Biosciences, Vienna, Austria) was added and the samples were incubated for 1 hour at room temperature, rinsed in TRIS buffer and processed for staining. Bound antibodies were visualized with DAB (3.3′diaminobenzidine) (Sigma, Vienna, Austria) as substrate chromogen. Slides were counterstained with Hemalaun and coverslipped using Eukitt (Merck, Darmstadt, Germany). Brightfield microscopy with photography was performed using a Leica DMRB microscope and a Nikon Coolpix 995 camera.

### EXAMPLE 9

### Immunohistochemical Detection of HPV-16 E7 in Prostate Derived Tissue

Biopsies were taken from 60 prostate carcinoma patients and samples from 60 patients were applied to an object slide together with negative controls. These slides are known to the expert as “tissue microarrays”. Tissue microarrays were stained with antibodies to HPV-16 E7 as described in FIG. 5 for cervical biopsies. In this experiment, a subset of the carcinoma biopsies stained positive for HPV-16 E7, whereas other biopsies from different prostate cancer patients were staining negative. In these experiments, HPV-16 E7 was detected in roughly 10% of the prostate carcinoma specimens analyzed. This result suggests that the subset of the prostate carcinomas express high levels of HPV-16 E7 and thereby provide evidence for a role of HPV-16 E7 in prostate carcinoma.

### EXAMPLE 10

### Detection of HPV-16 E7 (Onco-)Protein in Pre-Neoplastic and Neoplastic Cells from Ectocervical Smears (PapSmear)

To determine the presence of HPV-16 E7 protein in ectocervical smears (PapSmear, routinely used for cervical cancer screening), superficial cells were obtained by cervical smear examination (PapSmear) from women with normal cervical squamous epithelia (healthy control) and cervical squamous intraepithelial lesions. Biopsies were taken at the department for Gynecology and Obstetrics at the University Hospital in Innsbruck/Austria. Biopsies were taken from nine cytologically normal patients and from twenty patients with abnormal cytological appearance (classified as PapIII by the physician). Superficial cells were obtained by cervical smear examination (PapSmear) from women with normal cervical squamous epithelia (healthy control) and cervical squamous intraepithelial lesions. Cells were streaked out on a glass slide and air dried. Subsequently, cells were fixed in 5% H₂O₂ (freshly dissolved in absolute methanol) for 15 minutes. Before immunostaining the sections were washed twice in TRIS buffer (7.75 g Tris-HCl pH 7.5, 8.78 g NaCl ad 1 liter aqua dest) and processed for a 15 minutes blocking reaction in diluted (1:10 in TRIS buffer/1% BSA) goat-serum (DAKO, Hamburg, Germany). Sections were washed in TRIS/1% BSA buffer and incubated with the first antibody (affinity purified polyclonal rabbit anti-HPV-16 E7 antibody described herein) for 1 hour at room temperature in buffer B (10 mg/ml BSA /10 mg/ml NIH3T3 lysate in PBS). The samples were rinsed twice in TRIS/1% BSA buffer and incubated for 1 hour at room temperature with the second antibody (Biotin-conjugated anti-rabbit IgG, DAKO, Glostrup, Denmark). After the washing step in TRIS/1% BSA, ExtrAvidin-conjugated peroxidase solution (Amersham Biosciences, Vienna, Austria) was added and the samples were incubated for 1 hour at room temperature, rinsed in TRIS buffer and processed for staining. Bound antibodies were visualized with DAB (3.3′-diaminobenzidine) (Sigma, Vienna, Austria) as substrate chromogen. Slides were counterstained with Hemalaun and coverslipped using Eukitt (Merck, Darmstadt, Germany). Brightfield microscopy with photography was performed using a Leica DMRB microscope and a Nikon Coolpix 995 camera.

Smears were stained by immunohistochemistry using the affinity-purified anti-HPV-16E7 antibody described herein. A representative example is shown in FIG. 6: The antibodies did not stain superficial cells in cervical smear from normal HPV-DNA negative ectocervix (FIG. 6A, patient ID SM28961), only normal basophile (grey) superficial cells with normal nucleus-cytoplasm relation are visible in this smear. In parallel, a ectocervical smear from a patient (patient ID WM20276), that had been classified as PapIIID and which was typed as HPV-16 DNA positive by PCR analysis, was analyzed by immunohistochemistry. A biopsy taken from the patient one day later revealed CINIII phenotype; later immunohistochemical analysis demonstrated high level expression of HPV-16 E7 in the tumor cells. Only a few normal basophile (grey/blue) superficial cells with normal nucleus-cytoplasm relation can be recognized. However, roughly 50% of the cells show enlarged nucleus-cytoplasm relation. These so-called koilocytes are stained by the E7 antibodies as indicated by the brown colour. Only these cells are stained by the E7 antibodies but not the normal squamous epithelial cells and columnar epithelium cells (usually contained in ectocervical smears). This demonstrates that the anti-HPV-16 E7 antibodies described herein provide a highly specific and sensitive marker for the detection of abnormal precursor malignant cells in cervical smear preparations (Pap Smears).

### EXAMPLE 11

### Detection of HPV-16 E7 Protein in Pre-Neoplastic and Neoplastic Cells from Ectocervical Smears

According to Example 10, a further evaluation was carried out under local regulations in a blinded trial using patient material obtained at the University Hospital in Innsbruck/Austria. The evaluation was performed by experienced pathologists of the department of Gynecology and Obstetrics of the University Hospital Innsbruck/Austria who also validated the Pap smears and biopsies, respectively. For screening, Pap smears were taken from women with normal cervical squamous epithelia (healthy control) and cervical squamous intraepithelial lesions. Pap smears were collected with the consent of patients. In case of positive cytology, biopsies were taken at the department of Gynecology and Obstetrics at the University Hospital in Innsbruck/Austria.

From 23 women, two smears were taken: one for conservative examination (Papanicolao staining) and one for anti-HPV-16 E7 staining, using the affinity-purified anti-HPV-16 E7 antibody described herein, following the protocol described herein in example 10.

From the 23 Pap smear samples examined, 8 specimens had normal cytological appearance (classified as Pap II or lower, according to the Munich II classification (Soost H J. The Munich nomenclature; Recent Results Cancer Res 1993;133:105-11) and were tested by anti-HPV-16 E7 staining, using the affinity-purified anti-HPV-16 E7 antibody/serum of the invention.

In 15 specimens with abnormal cytological Pap smear appearance (classified either “higher than Pap II” or “Pap II, unclear”), HPV genotyping by PCR, conservative histomorphological examination of cervical tissue biopsy and anti-HPV-16 E7 staining, by using the affinity-purified anti-HPV-16 E7 antibody, was performed.

Excluding smears that were not assessable because of mucus or few cells, the results (Tab.1) show a clear correlation between an abnormal histology and the anti-HPV-16 E7 staining. As already demonstrated in Examples 10, it can be shown in the present invention that anti-HPV-16 E7 antibodies described herein provide a highly specific and sensitive marker for the detection of abnormal precursor and malignant cells in Pap smears.

TABLE 1
Anti-HPV-16 E7 antibodies in Pap smears

| | HPV-Type | | | Anti-HPV-16 E7 |
|---|---|---|---|---|
| Nr | PCR | Pap class | Histology | staining |
| 1 | 16 | rezid. Pap IIID/IV | CIN III | + |
| 2 | —*⁾ | rezid. Pap IIID | CIN I | + |
| 3 | 31 | Pap IV | CIN III | not assessable, |
| | | | | mucus |
| 4 | 33, 58 | rezid. Pap IIID | CIN I | not assessable, |
| | | | | mucus |
| 5 | 16 | Pap II unclear | CIN III | +/−, few cells |
| 6 | —*⁾ | Pap II unclear | CIN III | + |
| 7 | 16 | Pap IIID | PE Carcinoma | + |
| 8 | —*⁾ | Pap IIID | CIN I | + |
| 9 | —*⁾ | rezid. Pap IIID | CIN I | + |
| 10 | 39 (week) | Pap IV | CIN III | not assessable |
| 11 | 16 | Pap IV | CIN III | + |
| 12 | —*⁾ | Pap IIID | CIN I | + |
| 13 | 16 | Pap III | PE Carcinoma | few cells |
| 14 | 56 | Pap IV | CIN III | not assessable |
| 15 | 16 | rezid. Pap IIID | CIN II | + |
| 16 | ND | Pap II | ND | − |
| 17 | ND | Pap II | ND | − |
| 18 | ND | Pap II | ND | − |
| 19 | ND | Pap II | ND | − |
| 20 | ND | Pap II | ND | − |
| 21 | ND | Pap II | ND | − |
| 22 | ND | Pap II | ND | − |
| 23 | ND | Pap II | ND | − |

*⁾no HPV detectable
ND: not determined

### EXAMPLE 12

### Detection of HPV-16 E7 in Tissue Homogenates by Sandwich ELISA

Control of HPV-16 E7 Gene Expression in Biopsies by Western Blot

The expression level of the HPV-16 E7 protein was studied in biopsy material derived from HPV-16 DNA positive cervical carcinoma patients and in histologically normal tissue specimens obtained from patients (HPV-DNA negative by PCR) who underwent hysterectomy for diseases unrelated to the cervix uteri. Three HPV-16 DNA positive and seven unrelated cervical biopsies were analysed in Western blot experiments using the affinity purified anti-HPV-16 E7 antibodies. The specimens derived from HPV-16 DNA positive cervical carcinoma patients were all positive, whereas in the unrelated tissues E7 was not detectable (FIG. 1). In one biopsy (# 2424) the E7 protein level was as high as in CaSki cells, a cell line derived from a HPV-16 positive cervical carcinoma (Schwarz (1985 Nature 314, 111-119)). In the other HPV-16-positive specimens the E7 level was lower; however, the different expression levels in the individual biopsies can be explained by the fact that the portion of tumor material in a given biopsy differs. No signal was obtained with cervical cancer biopsies derived from HPV-45 positive patients.

16E7-ELISA for Detection of HPV-16 E7 in Liquid Samples Derived from Cervical Biopsies

To establish detection of HPV-16 E7 in liquid samples, 96-well ELISA plates were coated with IgG fractions derived from the polyclonal antibody described in the invention. Coated plates were incubated with crude lysates derived from E. coli-expressing HPV-16 E7 and control E. coli lysates. This experiment was used to determine the effective threshold value for reliable detection of 16 E7 antigen. To this end, affinity-purified anti-HPV-16 E7 antibodies were conjugated with horseradish peroxidase. The conjugate was added to the plate and after four washing steps, 3,3′,5,5′-tetra-methylbenzidine (TMB; Boehringer Mannheim # 784 974) was added. After incubation for one hour, conversion of TMB was analyzed by densitometric analysis at 450 nm, using a Dynatec ELISA reader. The values obtained for E. coli lysate were plotted relative to protein concentration and used to determine the threshold value for the absorption. The background value plus three standard deviations were used to calculate the threshold value above which a sample was considered E7-positive. In the present experiment, the threshold was set to A₄₅₀>0.16. In a second step, tissue homogenates of the human biopsies described above were prepared, diluted in ELISA buffer and analyzed by 16E7 ELISA, as described above. Results are shown in appended Table 2.

TABLE 2
Comparative analysis of HPV-16 E7 expression in tissue biopsies by 16E7
ELISA and Western blot
Biopsies from five cervical carcinoma patients
and biopsies from five hysterectomy patients without cervical
neoplasia (control) were analyzed for their content of HPV-16
E7 both by ELISA and Western blot techniques. The table gives
the absorption obtained in ELISA along with its evaluation
(cutoff A₄₅₀ > 0.16) and results obtained by
Western blot (see FIG. 1; grading derived from visual inspection).
Also indicated is the HPV DNA status of the patients, as determined
by PCR analysis.

| | 16 E7-ELISA | | | | |
|---|---|---|---|---|---|
| Biopsy # | A₄₅₀ | pos/neg | 16E7 Western | HPV DNA | comment |
| 1839 | 0.08 | negative | − | negative | control |
| 1867 | 0.02 | negative | − | negative | control |
| 2413 | 0.55 | positive | ++ | HPV-16 | |
| 3358 | 0.06 | negative | − | negative | control |
| 3366 | 0.09 | negative | − | negative | control |
| 2227 | 0.04 | negative | − | HPV-45 | |
| 2257 | 0.08 | negative | − | HPV-45 | |
| 2295 | 0.32 | positive | + | HPV-16 | |
| 2424 | 1.4 | positive | +++ | HPV-16 | |
| 2622 | 0.03 | negative | − | negative | control |

In the above described example 12, the following methods were employed

1.16 E7-ELISA

Coating

Affinity-purified polyclonal antibodies from rabbits immunized with highly purified native HPV-16 E7 proteins as described in this invention were precipitated by addition of ammonium sulfate to a final concentration of 45%, followed by centrifugation. After three consecutive precipitations, the antibody was dissolved in water, dialyzed 3× against ice-cold PBS and used at a final concentration of 2 μg per ml to coat ELISA plates (Nunc, Vienna).

Conjugation

2 mg of affinity-purified antibodies according to the invention were conjugated with horseradish peroxidase. Briefly antibodies at 2 mg/ml in PBS (1:10 diluted) were dialyzed overnight at 4 C against sodium carbonate buffer (0.01 M NaHCO3Na2CO3, pH 9.3). POD (Sigma cat. # P6782) was dissolved at 8 mg/ml in water and incubated with 1/10 volume of 0.2M NaJO4 for 20 min at RT in the dark. Subsequently, the POD solution was dialyzed overnight at 4 C against 1 mM sodium acetate/pH 4.4.

For coupling, the POD solution was adjusted to pH 9.3 and immediately incubated with the antibody solution. To this end, 650 μl antibody solution was added to 215 μl POD solution. The mixture was incubated at RT for 2 h under gentle agitation in the dark. To stop the reaction, 43 μl of Na(BH4) solution (4 mg/l aqua bidest.) was added and incubation continued for 2 h at RT. The conjugate was dialyzed against PBS overnight at 4 C, thiomersal was added to a final concentration of 0.1%. Conjugate was stored at 4 C.

Assay

After addition of TMB, peroxidase reaction and subsequent densitometric analyses in the ELISA reader were performed as described by the manufacturer.

2. E. coli Lysates

Construction of the Bacterial Expression Vector for HPV-16 E7

The HPV-16 E7 oncogene was amplified from the vector pX-HPV-16 E7 (Mannhardt (2000 Mol. Cell. Biol. 20, 6483-6495)) by PCR using Pfu DNA polymerase as Nde1/BamHI fragment. The sequence was inserted into the bacterial expression vector pET3a (Studier and Moffatt (1986 J. Mol. Biol. 189, 113-130)) prepared as NdeI/BamHI fragment generating the bacterial HPV-16 E7 expression vector pET3a-HPV-16 E7. The sequence encoding for HPV-16 E7 was verified by sequencing.

Preparation of Bacterial Lysates

The expression vector pET3a-HPV-16 E7/Clone 17 was transformed into E. coli strain BL21 (DE3) pLysS. The bacteria were grown to OD₆₀₀=0.5 and the expression of the E7 protein was induced for 3 hours at 37° C. by adding IPTG (Biomol, Hamburg, Germany) to a final concentration of 0.4 mM. For control lysates, the maternal strain E. coli (BL21 (DE3)pLysS) was used.

Bacteria were harvested by centrifugation for 10 minutes at 5 000×g. The cell pellets were frozen in 20 ml ice-cold lysis buffer (50 mM KCl, 20 mM H₂KPO₄ [pH 7.8], 50 mM DTT, 5% glycerol, 1 μg/ml leupeptin, 1 mM PMSF, 1 mM NaF and 10 μg/ml Aprotinin) per liter bacterial culture. Cells were thawed on ice and lysed by sonication with glass beads (Sigma, Vienna, Austria) using a Sonifier 250 (Branson, Geneva, Switzerland). After centrifugation at 70 000×g for 1 hour, the supernatant was ammonium sulfate precipitated using 30% saturated (NH₄)₂SO₄ solution. The (NH₄)₂SO4 pellet was dissolved in 150 mM Tris/Cl pH 7.8, 10 mM NaCl, dialysed against the same buffer and diluted to a final concentration of 10 mg/ml total protein.

3. Tissue Lysates

For protein extraction, biopsies were extracted in lysis buffer (10 mM Tris pH 7.5, 1% Triton X-100, 100 mM NAF, 0.2 mM PMSF). Samples are vortexed and redissolved by 20 strokes with a Branson sonifier on ice, followed by incubation on ice for 5 min. The sample is repeatedly frozen in liquid nitrogen, rethawed, and subsequently incubated on ice for another 15 minutes, followed by centrifugation for 30 min at 20.000 g. The supernatant is directly used for Western blot analysis or 16E7 ELISA.

4. Western Blot (Immunoblot) Analysis

Cell extracts were separated on a 12.5% sodium dodecyl sulfate (SDS)-polyacrylamide gel, and proteins were transferred to a PVDF membrane (NEN, Boston, USA). The membrane was incubated in blocking buffer (0.05% Tween 20/5% low fat milk powder in PBS) for 1 hour at room temperature, washed in blocking buffer and incubated with the first antibody (affinity purified polyclonal rabbit anti-HPV-16 E7 antibody) for 1 hour at room temperature. After washing in blocking buffer, PBS/0.05% Tween 20 and PBS/5% low fat milk powder the membrane was incubated with the second antibody (peroxidase-conjugated anti-rabbit IgG, Promega, Mannheim, Germany) for 45 minutes at room temperature. The membrane was washed, and the bound antibodies were visualized by using the chemiluminescence Western blotting detection system (NEN, Boston, USA).

### ADDITIONAL REFERENCES

Alexander and Phelps (2000) Expert Opin. Investig. Drugs 9: 1753-65

Bringold, (2000) Exp Gerontol 35:317-29

Clavel, (1999) Br J Cancer 80:1306-11

Francis, (2000) J Virol 74:2679-86

Fujikawa, (1994) Virology 204: 789-793

Giarre, (2001) J Virol 75:4705-12

Howley, (1996) Fields Virology, 3rd edn. Lipppincott-Raven, Philadelphia, Pa., pp. 2045-2076.

Klaes, (2001) Int J Cancer 92:276-84

Koss, (1989) J. Am. Med. Assoc. 261: 737-743

Larsen, N. S. (1994) J. Natl. Cancer Inst. 86: 6-7

McMurray, (2001) Int J Exp Pathol 82:15-33

Meijer, (2000) J Clin Pathol 53:41-3

Milde-Langosch, (2000) Virchows Arch 437:227-33

Milde-Langosch, (1999) Pathologe 20:15-24

Mantovani, (2001) Oncogene 20:7874-87

Münger, (2001) Oncogene 20:7888-98

Papanicolaou, (1942) Science 95: 438-439

Renshaw, (2001) Cancer 93:106-10

Sato, (1989) Virology: 170: 311-314

Schiffman, (1995) Cancer 76:1888-1901

Schwarz, (1985) Nature 314:111-114

Selvey (1992) J Virol Methods 37: 119-127

Stacey, (1994) Oncogene 9: 635-645

Studier, (1986) J Mol Biol 189:113-30

Tindle, (1990) Peptide Res 3:162-166

Trofatter, (1997) Jr. Am J Med 102:21-7

Serrano, (1997) Exp Cell Res 237:7-13

von Knebel Doeberitz, (2001) Dis Markers 17:123-8

Walboomers, (1995) J Clin Pathol 48:728-32

Walboomers, (1999) J Pathol 189:12-9

Zatsepina, (1997) Oncogene 14:1137-45

zur Hausen, (2000) J. Natl. Cancer Inst 92: 690-8

1. An anti-HPV-16 E7 antibody obtainable by
(a) eliciting an in vivo humoral response against highly purified HPV-16 E7 protein or a fragment thereof in a non-human vertebrate; and
(b) affinity-purifying antibodies as obtained in the eliciting-step (a).

2. The anti-HPV-16 E7 antibody of claim 1, wherein said highly purified HPV-16 E7 protein or a fragment thereof is recombinantly produced.

3. The anti-HPV-16 E7 antibody of claim 2, wherein said HPV-16 E7 protein or said fragment thereof is expressed in E. coli.

4. The anti-HPV-16 E7 antibody of claim 1, wherein said highly purified HPV-16 E7 protein or a fragment thereof is purified by a combination of ion exchange chromatography and gel filtration.

5. The anti-HPV-16 E7 antibody of claim 4, wherein said purification further comprises, prior to ion exchange chromatography and gel filtration, a protein precipitation step.

6. The anti-HPV-16 E7 antibody of claim 1, wherein said affinity purification of the obtained antibodies is carried out over immobilized HPV-16 E7 protein or a fragment thereof.

7. The anti-HPV-16 E7 antibody of claim 6, wherein said HPV-16 E7 protein or a fragment thereof is immobilized on PVDF membranes, nitrocellulose, sepharose, agarose, DEAE-cellulose or DEAE.

8. The anti-HPV-16 E7 antibody of claim 1, wherein said non-human vertebrate is selected from the group consisting of rat, mouse, rabbit, chicken, sheep, horse, goat, pig and donkey;

9.-11. (canceled)

12. A method for the preparation of a diagnostic composition comprising the step of formulating the anti-HPV-16 E7 antibody of claim 1 with a diagnostically acceptable carrier, diluent, buffer, or storage solution.

13. The method of claim 12, wherein said diagnostic composition further comprises suitable means for detection.

14. A diagnostic composition comprising the anti-HPV-16 E7 antibody of claim 1, optionally comprising a diagnostically acceptable carrier, diluent, buffer, or storage solution.

15. A kit comprising an anti-HPV-16 E7 of claim 1, or a diagnostic composition comprising the anti-HPV-16 E7 antibody with a diagnostically acceptable carrier, diluent, buffer, or storage solution, and optionally a suitable means for detection.

16. An in vitro method for the detection of a sexually transmittable disease or cancer comprising the steps of
a) incubating a biological sample with anti-HPV-16 E7 antibodies of claim 1; and
b) measuring and/or detecting specifically-bound anti-HPV-16 E7 antibodies whereby the presence, the absence or the amount of specifically-bound anti-HPV-16 E7 antibodies is indicative for said sexually transmittable disease or cancer.

17. The in vitro method of claim 16, further comprising a further step (c), whereby in said step (c) the presence, the absence or the amount of specifically-bound anti-HPV-16 E7 antibodies of step (b) is compared to the presence, the absence or the amount of specifically-bound anti-HPV-16 E7 antibodies in a negative or a positive control sample.

18. (canceled)

19. The in vitro method of claim 16, wherein said sexually transmitted disease is an HPV16-infection or wherein said cancer is cervical cancer, breast cancer/mamma cancer, prostate cancer, head and neck cancer, penil cancer and/or anogenital cancer/neoplasia (AIN).

20. A method for the production of an anti-HPV-16 E7 antibody comprising the steps of
(a) eliciting an in vivo humoral response against highly purified, HPV-16 E7 protein or a fragment thereof in a non-human vertebrate; and
(b) affinity-purifying antibodies as obtained in the eliciting-step (a).

21. The method of claim 20, wherein said highly purified HPV-16 E7 protein or said fragment thereof is a native, highly purified HPV-16 E7 protein or a fragment thereof.

22. The anti-HPV-16 E7 antibody of claim 1, wherein said highly purified HPV-16 E7 protein or said fragment thereof is a native, highly purified HPV-16 E7 protein or a fragment thereof.

23. The method of claim 16, wherein said biological sample is obtained from Pap-smears, cervical (carcinoma) biopsies, anogenital biopsies, mamma biopsies, head- or neck biopsies or prostate biopsies.

24. The method of claim 16, wherein said detection is used for evaluating the risk of acquiring a sexually transmitted disease or cancer, for measuring the status of an existing sexually transmitted disease or cancer, or for screening therapy efficiency in the treatment of a sexually transmitted disease or cancer.
